SEQUENCE LISTING (7 sequences)

SEQ ID NO: 1 - protein, 603 aa, Escherichia coli
MLRPVETPTREIKKLDGLWAFSLDRENCGIDQRWWESALQESRAIAVPGSFNDQFADADIRNYAGNVWYQREVFIPKGWAGQRIVLRFDAVTHYGKVWVNNQEVMEHQGGYTPFEADVTPYVIAGKSVRITVCVNNELNWQTIPPGMVITDENGKKKQSYFHDFFNYAGIHRSVMLYTTPNTWVDDITVVTHVAQDCNHASVDWQVVANGDVSVELRDADQQVVATGQGTSGTLQVVNPHLWQPGEGYLYELCVTAKSQTECDIYPLRVGIRSVAVKGEQFLINHKPFYFTGFGRHEDADLRGKGFDNVLMVHDHALMDWIGANSYRTSHYPYAEEMLDWADEHGIVVIDETAAVGFNLSLGIGFEAGNKPKELYSEEAVNGETQQAHLQAIKELIARDKNHPSVVMWSIANEPDTRPQGAREYFAPLAEATRKLDPTRPITCVNVMFCDAHTDTISDLFDVLCLNRYYGWYVQSGDLETAEKVLEKELLAWQEKLHQPIIITEYGVDTLAGLHSMYTDMWSEEYQCAWLDMYHRVFDRVSAVVGEQVWNFADFATSQGILRVGGNKKGIFTRDRKPKSAAFLLQKRWTGMNFGEKPQQGGKQ

SEQ ID NO: 2 - protein, 603 aa, Escherichia coli
MLRPVETPTREIKKLDGLWAFSLDRENCGIDQRWWESALQESRAIAVPGSFNDQFADADIRNYAGNVWYQREVFIPKGWAGQRIVLRFDAVTHYGKVWVNNQEVMEHQGGYTPFEADVTPYVIAGKSVRITVCVNNELNWQTIPPGMVITDENGKKKQSYFHDFFNYAGIHRSVMLYTTPNTWVDDITVVTHVAQDCNHASVDWQVVANGDVSVELRDADQQVVATGQGTSGTLQVVNPHLWQPGEGYLYELCVTAKSQTECDIYPLRVGIRSVAVKGEQFLINHKPFYFTGFGRHEDADLRGKGFDNVLMVHDHALMDWIGANSYRTSHYPYAEEMLDWADEHGIVVIDETAAVGFNLSLGIGFEAGNKPKELYSEEAVNGETQQAHLQAIKELIARDKNHPSVVMWSIANEPDTRPQGAREYFAPLAEATRKLDPTRPITCVNVMFCDAHTDTISDLFDVLCLNRYYGWYVQSGDLETAEKVLEKELLAWQEKLHQPIIITEYGVDTLAGLHSMYTDMWSEEYQCAWLDMYHRVFDRVSAVVGEQVWNFADFATSQSILRVGGNKKGIFTRDRKPKSAAFLLQKRWTGMNFGEKPQQGGKQ

SEQ ID NO: 3 - protein, 603 aa, Escherichia coli
MLRPVETPTREIKKLDGLWAFSLDRENCGIDQRWWESALQESRAIAVPGSFNDQFADADIRNYAGNVWYQREVFIPKGWAGQRIVLRFDAVTHYGKVWVNNQEVMEHQGGYTPFEADVTPYVIAGKSVRITVCVNNELNWQTIPPGMVITDENGKKKQSYFHDFFNYAGIHRSVMLYTTPNTWVDDITVVTHVAQDCNHASVDWQVVANGDVSVELRDADQQVVATGQGTSGTLQVVNPHLWQPGEGYLYELCVTAKSQTECDIYPLRVGIRSVAVKGEQFLINHKPFYFTGFGRHEDADLRGKGFDNVLMVHDHALMDWIGANSYRTSHYPYAEEMLDWADEHGIVVIDETAAVGFNLSLGIGFEAGNKPKELYSEEAVNGETQQAHLQAIKELIARDKNHPSVVMWSIANEPDTRPQGAREYFAPLAEATRKLDPTRPITCVNVMFCDAHTDTISDLFDVLCLNRYYGWYVQSGDLETAEKVLEKELLAWQEKLHQPIIITEYGVDTLAGLHSMYTDMWSEEYQCAWLDMYHRVFDRVSAVVGEQVWNFADFATSQTILRVGGNKKGIFTRDRKPKSAAFLLQKRWTGMNFGEKPQQGGKQ

SEQ ID NO: 4 - protein, 606 aa, Escherichia coli
MLRPVETPTREIKKLDGLWAFSLDRENCGIDQRWWESALQESRAIAVPGSFNDQFADADIRNYAGNVWYQREVFIPKGWAGQRIVLRFDAVTHYGKVWVNNQEVMEHQGGYTPFEADVTPYVIAGKSVRITVCVNNELNWQTIPPGMVITDENGKKKQSYFHDFFNYAGIHRSVMLYTTPNTWVDDITVVTHVAQDCNHASVDWQVVANGDVSVELRDADQQVVATGQGTSGTLQVVNPHLWQPGEGYLYELCVTAKSQTECDIYPLRVGIRSVAVKGEQFLINHKPFYFTGFGRHEDADLRGKGFDNVLMVHDHALMDWIGANSYRTSHYPYAEEMLDWADEHGIVVIDETAAVGFNLSLGIGFEAGNKPKELYSEEAVNGETQQAHLQAIKELIARDKNHPSVVMWSIANEPDTRPQGAREYFAPLAEATRKLDPTRPITCVNVMFCDAHTDTISDLFDVLCLNRYYGWYVQSGDLETAEKVLEKELLAWQEKLHQPIIITEYGVDTLAGLHSMYTDMWSEEYQCAWLDMYHRVFDRVSAVVGEQVWNFADFATSQSILRVGGNKKGIFTRDRKPKSAAFLLQKRWTGMNFGEKPQQGGKQGLC

SEQ ID NO: 5 - protein, 600 aa, Escherichia coli
MLRPVETPTREIKKLDGLWAFSLDRENCGIDQRWWESALQESRAIAVPGSFNDQFADADIRNYAGNVWYQREVFIPKGWAGQRIVLRFDAVTHYGKVWVNNQEVMEHQGGYTPFEADVTPYVIAGKSVRITVCVNNELNWQTIPPGMVITDENGKKKQSYFHDFFNYAGIHRSVMLYTTPNTWVDDITVVTHVAQDCNHASVDWQVVANGDVSVELRDADQQVVATGQGTSGTLQVVNPHLWQPGEGYLYELCVTAKSQTECDIYPLRVGIRSVAVKGEQFLINHKPFYFTGFGRHEDADLRGKGFDNVLMVHDHALMDWIGANSYRTSHYPYAEEMLDWADEHGIVVIDETAAVGFNLSLGIGPKELYSEEAVNGETQQAHLQAIKELIARDKNHPSVVMWSIANEPDTRPQGAREYFAPLAEATRKLDPTRPITCVNVMFCDAHTDTISDLFDVLCLNRYYGWYVQSGDLETAEKVLEKELLAWQEKLHQPIIITEYGVDTLAGLHSMYTDMWSEEYQCAWLDMYHRVFDRVSAVVGEQVWNFADFATSQSILRVGGNKKGIFTRDRKPKSAAFLLQKRWTGMNFGEKPQQGGKQGLC

SEQ ID NO: 6 - protein, 594 aa, Escherichia coli
MLRPVETPTREIKKLDGLWAFSLDRENCGIDQRWWESALQESRAIAVPGSFNDQFADADIRNYAGNVWYQREVFIPKGWAGQRIVLRFDAVTHYGKVWVNNQEVMEHQGGYTPFEADVTPYVIAGKSVRITVCVNNELNWQTIPPGMVITDENGKKKQSYFHDFFNYAGIHRSVMLYTTPNTWVDDITVVTHVAQDCNHASVDWQVVANGDVSVELRDADQQVVATGQGTSGTLQVVNPHLWQPGEGYLYELCVTAKSQTECDIYPLRVGIRSVAVKGEQFLINHKPFYFTGFGRHEDADLRGKGFDNVLMVHDHALMDWIGANSYRTSHYPYAEEMLDWADEHGIVVIDETAAVGFNLSLGIGEEAVNGETQQAHLQAIKELIARDKNHPSVVMWSIANEPDTRPQGAREYFAPLAEATRKLDPTRPITCVNVMFCDAHTDTISDLFDVLCLNRYYGWYVQSGDLETAEKVLEKELLAWQEKLHQPIIITEYGVDTLAGLHSMYTDMWSEEYQCAWLDMYHRVFDRVSAVVGEQVWNFADFATSQSILRVGGNKKGIFTRDRKPKSAAFLLQKRWTGMNFGEKPQQGGKQGLC

SEQ ID NO: 7 - protein, 594 aa, Escherichia coli
MLRPVETPTREIKKLDGLWAFSLDRENCGIDQRWWESALQESRAIAVPGSFNDQFADADIRNYAGNVWYQREVFIPKGWAGQRIVLRFDAVTHYGKVWVNNQEVMEHQGGYTPFEADVTPYVIAGKSVRITVCVNNELNWQTIPPGMVITDENGKKKQSYFHDFFNYAGIHRSVMLYTTPNTWVDDITVVTHVAQDCNHASVDWQVVANGDVSVELRDADQQVVATGQGTSGTLQVVNPHLWQPGEGYLYELCVTAKSQTECDIYPLRVGIRSVAVKGEQFLINHKPFYFTGFGRHEDADLRGKGFDNVLMVHDHALMDWIGANSYRTSHYPYAEEMLDWADEHGIVVIDETAAVGFNLSLGIGPQFFNGETQQAHLQAIKELIARDKNHPSVVMWSIANEPDTRPQGAREYFAPLAEATRKLDPTRPITCVNVMFCDAHTDTISDLFDVLCLNRYYGWYVQSGDLETAEKVLEKELLAWQEKLHQPIIITEYGVDTLAGLHSMYTDMWSEEYQCAWLDMYHRVFDRVSAVVGEQVWNFADFATSQSILRVGGNKKGIFTRDRKPKSAAFLLQKRWTGMNFGEKPQQGGKQGLC

(19) United States
(12) Patent Application Publication
(10) Pub. No.: US 2020/0024586 A1
(43) Pub. Date: Jan. 23, 2020

(54) THERMOSTABILE BETA-GLUCURONIDASE FORMULATIONS

(71) Applicant: INTEGRATED MICRO-CHROMATOGRAPHY SYSTEMS, INC., Irmo, SC (US)

(72) Inventors: Lim Andrew LEE, Columbia, SC (US); Pongkwan Nikki SITASUWAN, Columbia, SC (US); Margarita MARINOVA, Columbia, SC (US)

(73) Assignee: INTEGRATED MICRO-CHROMATOGRAPHY SYSTEMS, INC., Irmo, SC (US)

(21) Appl. No.: 16/478,674

(22) PCT Filed: Jan. 20, 2017
(86) PCT No.: PCT/US2017/014387

§ 371 Date: Jul. 17, 2019

(51) Int. Cl. C12N 9/24; C12Q 1/40

(52) U.S. Cl. CPC C12N 9/2402; C12Q 1/40; C12Y 302/01031

(57) ABSTRACT

Formulations of β-glucuronidase enzymes that exhibit long-term stability at both high and low temperatures as either aqueous or lyophilized formulations are provided. The formulations of the invention comprise a β-glucuronidase enzyme, such as a mutant β-glucuronidase enzyme, an amphoteric compound, L-histidine,β-alanine and a sugar. Methods of preparing the formulations, as well as methods of using the formulations for hydrolysis of glucuronide substrates, including opiates and benzodiazepines, are also provided.

### BACKGROUND OF THE INVENTION

In mammals, glucuronidation is one of the principle means of detoxifying or inactivating compounds using the UDP glucuronyl transferase system. Compounds are conjugated by the glucoronyl transferase system to form glucuronides, which are then secreted in urine or into the lower intestine in bile. The β-glucuronidase (BGUS) enzyme catalyzes the hydrolysis of a wide variety of β-glucuronides. Given the key role of glucuronidation in detoxification of compounds, the BGUS enzyme has been used for detection of drugs in bodily samples, such as to detect the presence of illicit drugs in bodily samples of criminal suspects. For example, a bodily sample can be tested for the presence of a suspected drug by detecting the hydrolysis of the glucuronide form of the drug by BGUS. The hydrolysis of the glucuronic acid by the BGUS enzyme facilitates the analysis of the drug by methods such as mass spectrometry, since this analytical instrument is less sensitive in the presence of glucuronic acid.

Commercially available preparations of BGUS enzyme typically are provided as aqueous formulations and may not exhibit enzymatic stability over a wide temperature range or for prolonged periods of time. Accordingly, there is a need for BGUS enzyme formulations with enhanced thermostability properties that are suitable both for aqueous and lyophilized formulations.

### SUMMARY OF THE INVENTION

The invention provides BGUS enzyme formulations that exhibit thermostability at both low temperatures (e.g., below freezing) and high temperatures (e.g., above 37 degrees C.) as either a liquid formulation or as a lyophilized formulation. Thus, these formulations permit freeze/thawing while maintaining enzymatic activity. Moreover, the formulations described herein exhibit enzymatic activity for prolonged periods (e.g., greater than 10 days) at elevated temperatures (>37 degrees C.) and have low propensity for aggregation, thus providing a long storage life. Still further, the formulations are free of excipients such as detergents and polymers that can interfere with use of the enzyme in certain types of assays (e.g., mass spectrometry). Thus, the thermostable formulations of the invention can be used in the direct analysis of biological samples (e.g., urine samples) with minimal cleanup.

Based on analysis of a large panel of amino acids, sugars, and salts, it has been discovered that formulations containing an amphoteric compound, the amino acids L-histidine and β-alanine, and a sugar exhibit the desirable thermostability and other advantageous properties discussed above. Accordingly, in one aspect, the invention pertains to a formulation comprising a β-glucuronidase (BGUS) enzyme, an amphoteric compound, L-histidine, β-alanine and a sugar.

In one embodiment, the amphoteric compound is selected from the group consisting of betaine monohydrate, choline salts, betaine salts, 6-aminohexanoic acid, 5-aminovaleric acid and 4-aminobutyric acid (GABA). In one embodiment the amphoteric compound is betaine monohydrate. In one embodiment, betaine monohydrate is present in the formulation at a concentration of at least 50 mM. In another embodiment, betaine monohydrate is present in the formulation at a concentration of 50 mM-250 mM. In yet another embodiment, betaine monohydrate is present in the formulation at a concentration of 250 mM. Additional suitable concentrations are disclosed herein.

In one embodiment, the sugar is selected from the group consisting of sucrose, sorbitol, xylitol, glycerol, 2-hydroxypropyl-β-cyclodextrin and α-cyclodextrin. In one embodiment, the sugar is sucrose. In one embodiment, sucrose is present in the formulation at a concentration of at least 50 mM. In another embodiment, sucrose is present in the formulation at a concentration of 50 mM-500 mM. In yet another embodiment, sucrose is present in the formulation at a concentration of 500 mM. Additional suitable concentrations are disclosed herein.

In one embodiment, β-alanine is present in the formulation at a concentration of at least 50 mM. In another embodiment, β-alanine is present in the formulation at a concentration of 50 mM-250 mM. In yet another embodiment, β-alanine is present in the formulation at a concentration of 250 mM. Additional suitable concentrations are disclosed herein.

In one embodiment, L-histidine is present in the formulation at a concentration of at least 10 mM. In another embodiment, L-histidine is present in the formulation at a concentration of 10 mM-50 mM. In yet another embodiment, L-histidine is present in the formulation at a concentration of 50 mM. Additional suitable concentrations are disclosed herein.

In one embodiment, the BGUS enzyme in the formulation is a recombinant BGUS enzyme. In one embodiment, the recombinant BGUS enzyme is a mutant BGUS enzyme. In one embodiment, the mutant BGUS enzyme is a mutant E. coli BGUS enzyme, such as an enzyme having the amino acid sequence shown in SEQ ID NO: 2 or SEQ ID NO: 3. Additional suitable BGUS enzymes for use in the formulation are disclosed herein.

In one embodiment, the BGUS enzyme is present in the formulation at a concentration of at least 1 mg/ml. In another embodiment, the BGUS enzyme is present in the formulation at a concentration of 1-5 mg/ml. In yet another embodiment, the BGUS enzyme is present in the formulation at a concentration of 5 mg/ml. Additional suitable concentrations are disclosed herein.

In one embodiment, the formulation is free of detergents. In another embodiment, the formulation is free of polymers.

In one embodiment, the formulation is an aqueous formulation. In another embodiment, the formulation is a lyophilized formulation.

In one embodiment, the formulation comprises a β-glucuronidase (BGUS) enzyme, 50 mM betaine monohydrate, 10 mM L-histidine, 50 mM β-alanine and 50 mM sucrose. In one embodiment, this formulation further comprises a BGUS enzyme having the amino acid sequence shown in SEQ ID NO: 2 or SEQ ID NO: 3 at a concentration of 1 mg/ml. In one embodiment, the formulation is an aqueous formulation. Alternatively, this formulation can be a lyophilized formulation.

In one embodiment, the formulation comprises a β-glucuronidase (BGUS) enzyme, 250 mM betaine monohydrate, 50 mM L-histidine, 250 mM β-alanine and 250 mM sucrose. In one embodiment, this formulation further comprises a BGUS enzyme having the amino acid sequence shown in SEQ ID NO: 2 or SEQ ID NO: 3 at a concentration of 5 mg/ml. In one embodiment, the formulation is a lyophilized formulation. Alternatively, this formulation can be an aqueous formulation.

In another aspect, the invention provides methods of preparing the formulations of the invention. Accordingly, in one embodiment, the invention provides a method of preparing a β-glucuronidase (BGUS) enzyme formulation comprising:

(a) preparing a solution comprising a BGUS enzyme, an amphoteric compound, L-histidine and β-alanine; and

(b) adding a sugar to the solution prepared in step (a).

In on embodiment, the method further comprises lyophilizing the solution prepared in step (b).

In yet another aspect, the invention pertains to concentrated BGUS enzyme formulations, such as lyophilized formulations, that are to be diluted (e.g., with water) prior to use in an enzymatic assay. Accordingly, in one embodiment, the invention provides a packaged β-glucuronidase (BGUS) enzyme formulation comprising a container comprising a lyophilized formulation comprising 5 mg/ml BGUS enzyme, 250 mM betaine monohydrate, 50 mM L-histidine, 250 mM β-alanine and 250 mM sucrose; and instructions to dilute the lyophilized formulation to 1 mg/ml BGUS enzyme, 50 mM betaine monohydrate, 10 mM L-histidine, 50 mM β-alanine and 50 mM sucrose before use in an enzymatic assay.

In yet another aspect, the invention pertains to methods of using the formulations of the invention to hydrolyze a glucuronide linkage, such as in drug testing of a biological sample. Accordingly, in one aspect, the invention pertains to a method of hydrolyzing a substrate comprising a glucuronide linkage, the method comprising contacting the substrate with a formulation of the invention under conditions such that hydrolysis of the glucuronide linkage occurs. In one embodiment, the substrate is an opiate glucuronide. Suitable opiate glucuronides are disclosed herein. In another embodiment, the substrate is a benzodiazepine glucuronide. Suitable benzodiazepine glucuronides are disclosed herein. In one embodiment, the substrate is in a biological sample, such as blood, urine, tissue or meconium, obtained from a subject.

Other features and aspects of the invention are described in further detail herein.

### BRIEF DESCRIPTION OF THE DRAWINGS

FIG. 1 is an alignment of the amino acid sequences of the K1S (SEQ ID NO: 2), K1T (SEQ ID NO: 3), K3 (SEQ ID NO: 4), K3Δ1 (SEQ ID NO: 5), K3Δ2 (SEQ ID NO: 6) and K3Δ2S (SEQ ID NO: 7) mutants as compared to the wild type E. coli K12 sequence (SEQ ID NO: 1). The F385 through S396 modification region, G559S or G559T modifications and C-terminal GLC modification in the mutants are highlighted in bold and underlined.

### DETAILED DESCRIPTION OF THE INVENTION

The invention pertains to β-glucuronidase (BGUS) enzyme formulations having enhanced thermostability properties, as well as additional advantageous properties. The formulations of the invention can be either liquid (aqueous) or lyophilized (freeze-dried). The liquid formulations of the invention allow for maintenance of enzymatic activity even after cycles of freezing/thawing. The lyophilized formulations of the invention maintain enzymatic activity over a wide temperature range, including high temperatures (e.g., above 37 degrees C.). For example, storage of the lyophilized enzyme formulation at elevated temperatures of 40 or 50 degrees C. was permissible up to 5 days with no loss of enzyme activity.

As discussed in detail in the Examples, a large panel of amino acids, sugars and salts, and combinations thereof, were tested to identify compounds that enhanced the stability of the BGUS enzyme over a wide range of temperatures, while maintaining enzymatic activity over time and avoiding aggregation in the formulation. These experiments identified formulations containing an amphoteric compound (e.g., betaine), L-histidine, β-alanine and a sugar (e.g., sucrose) as providing the most desirable thermostability properties while maintaining enzymatic activity and avoiding aggregation.

Various aspects of the invention are described in further detail in the following subsections.

### I. Aqueous and Lyophilized Formulations

The formulations of the invention comprise a β-glucuronidase (BGUS) enzyme, an amphoteric compound, L-histidine, β-alanine and a sugar. As used herein, an “amphoteric compound” refers to a substance that has the ability to act either as an acid or a base. Suitable amphoteric compounds include betaine monohydrate (CAS No. 590-47-6; also referred to herein simply as “betaine”), 6-aminohexanoic acid (CAS No. 60-32-2), 5-aminovaleric acid (CAS No. 660-88-8) and 4-aminobutyric acid (GABA) (CAS No. 56-12-2). Additional amphoteric compounds include choline compounds, including choline acetate (CAS No. 14586-35-7), choline hydroxide (CAS No. 123-41-1) and choline chloride (CAS No. 67-48-1). In one embodiment, the amphoteric compound is selected from the group consisting of betaine monohydrate, choline salts, betaine salts, 6-aminohexanoic acid, 5-aminovaleric acid and 4-aminobutyric acid (GABA). In a preferred embodiment, the amphoteric compound is betaine monohydrate.

In certain embodiments, the amphoteric compound (e.g., betaine) is present in the formulation at a concentration of at least 10 mM, or at least 25 mM or at least 50 mM. In other embodiments, the amphoteric compound (e.g., betaine) is present in the formulation at a concentration of 10-500 mM, or 25-500 mM or 50 mM-250 mM. In other embodiments, the amphoteric compound (e.g., betaine) is present in the formulation at a concentration of 10 mM or 20 mM or 25 mM or 30 mM or 40 mM or 50 mM or 75 mM or 100 mM or 200 mM or 250 mM or 300 mM or 400 mM or 500 mM.

Alternative to use of an amphoteric compound in a formulation of the invention, amine oxides such as trimethylamine N-oxide also are suitable for use as a thermostabilizing agent in the enzyme formulations instead of the amphoteric compound. However, given the significantly higher cost of such compounds as compared to the cost of the amphoteric compounds, use of amine oxides such as trimethylamine N-oxide are less preferred for use in the formulations.

In certain embodiments, the sugar used in the formulation is a polyol. In certain embodiments, the sugar used in the formulation is selected from the group consisting of sucrose, sorbitol, xylitol, glycerol, 2-hydroxypropyl-β-cyclodextrin and α-cyclodextrin. In a preferred embodiment, the sugar is sucrose.

In certain embodiments, the sugar (e.g., sucrose) is present in the formulation at a concentration of at least 10 mM, or at least 25 mM or at least 50 mM or at least 100 mM. In other embodiments, the sugar (e.g., sucrose) is present in the formulation at a concentration of 10-1000 mM, or 25-500 mM or 50 mM-250 mM or 50 mM-500 mM or 50 mM-1000 mM. In other embodiments, the sugar (e.g., sucrose) is present in the formulation at a concentration of 50 mM or 75 mM or 100 mM or 200 mM or 250 mM or 300 mM or 400 mM or 500 mM or 600 mM or 700 mM or 750 mM or 800 mM or 900 mM or 1000 mM.

In certain embodiments, β-alanine is present in the formulation at a concentration of at least 25 mM or at least 50 mM. In other embodiments, β-alanine is present in the formulation at a concentration of 25-500 mM or 50 mM-250 mM or 50 mM-500 mM. In other embodiments, β-alanine is present in the formulation at a concentration of 25 mM or 30 mM or 40 mM or 50 mM or 75 mM or 100 mM or 200 mM or 250 mM or 300 mM or 400 mM or 500 mM.

In certain embodiments, L-histidine is present in the formulation at a concentration of at least 10 mM or at least 15 mM or at least 20 mM or at least 25 mM. In other embodiments, L-histidine is present in the formulation at a concentration of 10-100 mM or 10 mM-50 mM or 10 mM-25 mM. In other embodiments, L-histidine is present in the formulation at a concentration of 10 mM or 15 mM or 20 mM or 25 mM or 30 mM or 35 mM or 40 mM or 45 mM or 50 mM or 75 mM or 100 mM.

Suitable BGUS enzymes for use in the formulations are described further in subsection II below. In certain embodiments, the BGUS enzyme is present in the formulation at a concentration of at least 1 mg/ml or at least 2.5 mg/ml or at least 5 mg/ml or at least 10 mg/ml. In other embodiments, the BGUS enzyme is present in the formulation at a concentration of 1-10 mg/ml or 1-5 mg/ml mM or 2.5-10 mg/ml or 2.5-5 mg/ml. In other embodiments, the BGUS enzyme is present in the formulation at a concentration of 1 mg/ml or 2 mg/ml or 3 mg/ml or 4 mg/ml or 5 mg/ml or 6 mg/ml or 7 mg/ml or 8 mg/ml or 9 mg/ml or 10 mg/ml.

In certain embodiments, the BGUS enzyme in the formulation has an enzymatic activity of at least 5,000 Units/ml or 5,000 Units/mg, more preferably at least 10,000 Units/ml or 10,000 Units/mg, even more preferably at least 25,000 Units/ml or 25,000 Units/mg and even more preferably 50,000 Units/ml or 50,000 Units/mg. In one embodiment, the β-glucuronidase enzyme in the preparation is in an aqueous solution with an enzymatic activity of at least 5,000 Units/ml, or at least 10,000 Units/ml or at least 25, 000 Units/ml or at least 50,000 Units/ml. In another embodiment, the β-glucuronidase enzyme in the preparation is in lyophilized form with an enzymatic activity of at least 5,000 Units/mg, or at least 10,000 Units/mg or at least 25, 000 Units/mg or at least 50,000 Units/mg. In yet another embodiment, the β-glucuronidase enzyme in the preparation is in lyophilized form that when reconstituted as an aqueous solution has an enzymatic activity of at least 5,000 Units/ml, or at least 10,000 Units/ml or at least 25,000 Units/ml or at least 50,000 Units/ml.

The specific activity of the enzyme in the preparation, in Units/ml or Units/mg, can be determined using a standardized glucuronide linkage hydrolysis assay using phenolphthalein-glucuronide as the substrate. The standardization of the specific activity of BGUS has been well established in the art. Thus, 1 Fishman unit of BGUS activity is defined as an amount of enzyme that liberates 1 μg of phenolphthalein from phenolphthalein-glucuronide in 1 hour. An exemplary standardized assay that can be used to determine the specific activity (in Units/ml or Units/mg) of an enzyme preparation (e.g., an aqueous solution or lyophilized preparation) is described in further detail in Example 5. The skilled artisan will appreciate that other protocols for the enzyme assay are also suitable (e.g., such as those described by Sigma Aldrich Chemical Co.).

In one embodiment, the formulation is free of detergents, such as surfactants (e.g., Tween compounds and the like). Since the presence of detergents in a BGUS formulation can interfere with mass spectrometry (MS) analysis, the lack of detergent(s) in the formulation of the invention imparts the advantage that the formulation can be used directly in analysis of biological samples to be assayed by MS.

In one embodiment, the formulation is free of polymers (e.g., synthetic polymers and the like). Since the presence of polymers in a BGUS formulation can interfere with mass spectrometry (MS) analysis, the lack of polymer(s) in the formulation of the invention imparts the advantage that the formulation can be used directly in analysis of biological samples to be assayed by MS.

In one embodiment, the formulation is an aqueous (liquid) formulation. In another embodiment, the formulation is a lyophilized (freeze-dried) formulation. Methods for preparing the aqueous and lyophilized formulations are described further in subsection III below. With respect to the concentration of the BGUS enzyme and excipients in lyophilized formulations, the indicated concentration of BGUS enzyme in mg/ml and the indicated concentration of excipients in mM recited herein refers to the concentrations of BGUS enzyme and excipients in the starting aqueous solution used to make the lyophilized formulation.

In alternative embodiments, the formulation comprises a BGUS enzyme, an amphoteric compound, L-histidine and β-alanine. In alternative embodiments, the formulation comprises a BGUS enzyme, an amphoteric compound, L-histidine and a sugar. In alternative embodiments, the formulation comprises a BGUS enzyme, an amphoteric compound, β-alanine and a sugar. In alternative embodiments, the formulation comprises a BGUS enzyme, L-histidine, β-alanine and a sugar. In alternative embodiments, the formulation comprises a BGUS enzyme, an amphoteric compound and a sugar. In alternative embodiments, the formulation comprises a BGUS enzyme, an amphoteric compound and L-histidine. In alternative embodiments, the formulation comprises a BGUS enzyme, an amphoteric compound and β-alanine. In alternative embodiments, the formulation comprises a BGUS enzyme, L-histidine and β-alanine. In alternative embodiments, the formulation comprises a BGUS enzyme, L-histidine and a sugar. In alternative embodiments, the formulation comprises a BGUS enzyme, β-alanine and a sugar. Suitable components for these alternative embodiments are as described above.

In one embodiment, the formulation comprises a β-glucuronidase (BGUS) enzyme, 50 mM betaine monohydrate, 10 mM L-histidine, 50 mM β-alanine and 50 mM sucrose. Suitable BGUS enzymes for use in the formulation are described in subsection II below. In one embodiment, the BGUS enzyme is a mutant E. coli BGUS enzyme. In one embodiment, the BGUS enzyme has the amino acid sequence shown in SEQ ID NO: 2 or SEQ ID NO: 3. In one embodiment, the BGUS enzyme is present in the formulation at a concentration of 1 mg/ml. In one embodiment the formulation is an aqueous formulation. Alternatively, the formulation can be a lyophilized formulation.

In one embodiment, the formulation comprises a β-glucuronidase (BGUS) enzyme, 250 mM betaine monohydrate, 50 mM L-histidine, 250 mM β-alanine and 250 mM sucrose. Suitable BGUS enzymes for use in the formulation are described in subsection II below. In one embodiment, the BGUS enzyme is a mutant E. coli BGUS enzyme. In one embodiment, the BGUS enzyme has the amino acid sequence shown in SEQ ID NO: 2 or SEQ ID NO: 3. In one embodiment, the BGUS enzyme is present in the formulation at a concentration of 5 mg/ml. In one embodiment the formulation is a lyophilized formulation. Alternatively, the formulation can be an aqueous formulation.

The formulations of the invention exhibit enzymatic stability over a broad range of temperatures and for prolonged periods of time. As used herein, a formulation being “stable” refers to the BGUS enzyme in the formulation maintaining at least 80%, more preferably at least 90%, even more preferably at least 95% of its enzymatic activity over the indicated time and/or at the indicated temperature. In one embodiment, an aqueous formulation of the invention remains stable after one or more cycles of freezing/thawing (e.g., freezing at 4 degrees C. and thawing at 20 degrees C.). In one embodiment, an aqueous formulation of the invention remains stable after 1-3 cycles of freezing/thawing. In one embodiment, an aqueous formulation of the invention remains stable for at least one month, more preferably at least three months, and even more preferably at least six months at 2-8° C. (e.g., at 4 degrees C.). In one embodiment, an aqueous formulation of the invention remains stable for at least 7 days, more preferably at least 14 days, and even more preferably at least 21 days at 20° C. In one embodiment, an aqueous formulation of the invention remains stable for at least 7 days, more preferably at least 14 days, and even more preferably at least 21 days at 37° C. In one embodiment, a lyophilized formulation of the invention remains stable for at least 24 hours at 40° C. or for at least 48 hours at 37° C. or for least 5 days at 37° C.

### II. BGUS Enzymes

As used herein, the term “β-glucuronidase enzyme”, also referred to as “β-glucuronidase” or “BGUS”, refers to an enzyme that hydrolyzes β-glucuronide linkages. A BGUS enzyme used in a formulation of the invention can be, for example, a wild type enzyme or a mutated enzyme. A “wild type” BGUS enzyme refers to the naturally occurring form of the enzyme. A “mutated” BGUS enzyme refers to a modified form of the enzyme in which one or more modifications, such as amino acid substitutions, deletions and/or insertions, have been made such that the amino acid sequence of the mutated BGUS enzyme differs from the wild type amino acid sequence. A BGUS enzyme used in a formulation of the invention can be, for example, a recombinant BGUS enzyme. A “recombinant” BGUS enzyme refers to a genetically engineered form of a BGUS enzyme, as opposed to an enzyme that has been extracted from a natural biological source (e.g., extracted from bacteria, snails or abalone).

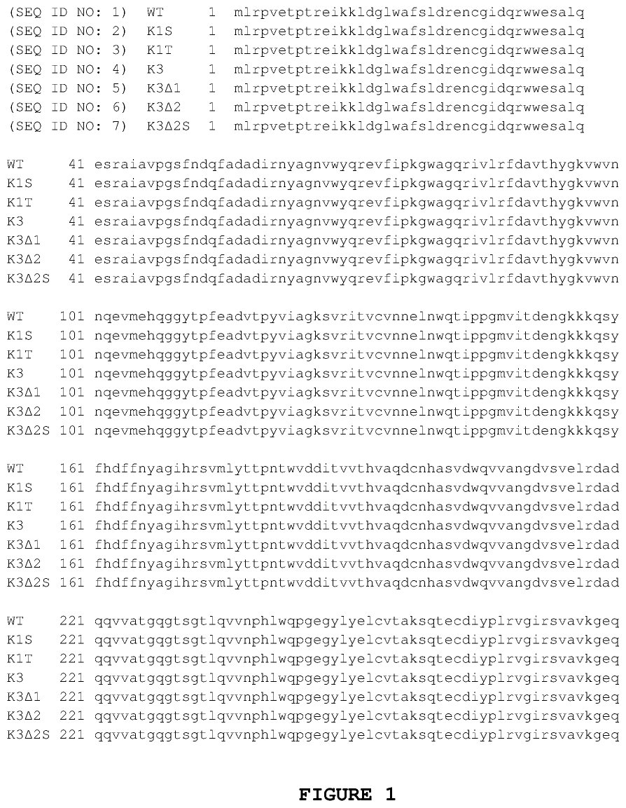

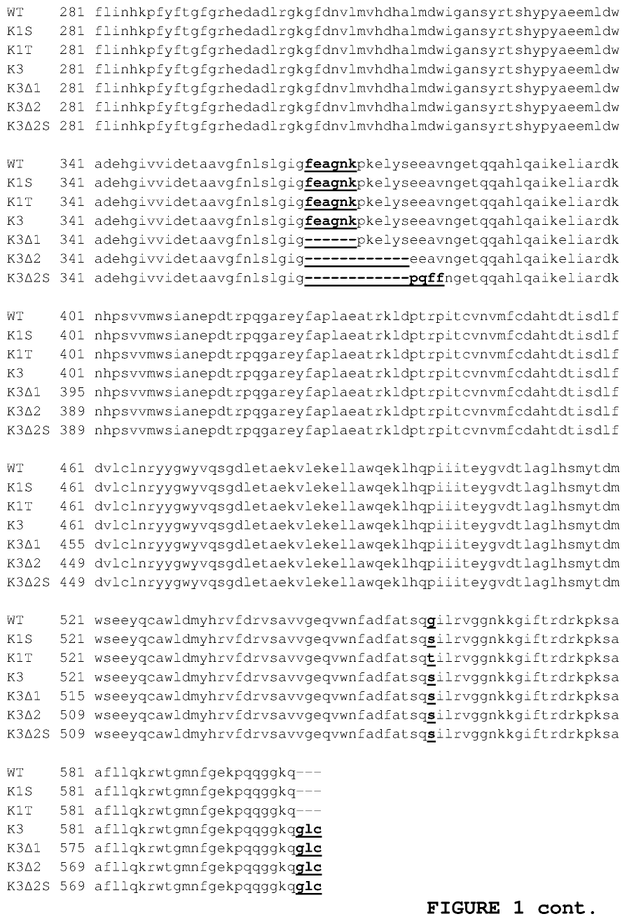

The sequences of wild type BGUS enzymes from numerous species are known in the art. For example, the nucleotide sequence encoding wild type E. coli K12 strain BGUS is shown in NCBI Reference Sequence: NC_000913.2 and the amino acid sequence of wild type E. coli K12 strain BGUS is shown in FIG. 1 and SEQ ID NO: 1. The amino acid sequence of wild type human (Homo sapiens) BGUS (isoform 1 precursor) is shown in NCBI Reference Sequence NP_000172.2. The amino acid sequence of wild type mouse (Mus musculus) BGUS (precursor) is shown NCBI Reference Sequence NP_034498.1. The amino acid sequence of wild type Lactobacillus brevis BGUS is shown in Genbank Accession No. ACU21612.1. The amino acid sequence of wild type Staphylococcus sp. RLH1 BGUS is shown in Genbank Accession No. AAK29422.1. Furthermore, the sequences of a number of microbial BGUS enzymes are disclosed in U.S. Pat. No. 6,391,547 and EP Patent EP 1175495B, the entire contents of which, including the sequence listing, are incorporated herein by reference.

The sequences of mutant BGUS enzymes from numerous species are known in the art. Suitable mutant BGUS enzymes are disclosed in, for example, U.S. Patent Publication 2016/0090582, the entire contents of which, including the sequences, is expressly incorporated herein by reference. Additional mutant BGUS enzymes have been described in the art, e.g., in Xiong, A-S. et al. (2007) Prot. Eng. Design Select. 20:319-325. Furthermore, a mutant BGUS enzyme suitable for use in the invention is commercially available (IMCSzyme®, Integrated Micro-Chromatography Systems, LLC).

In one embodiment, the mutant BGUS enzyme is a mutant E. coli K12 strain BGUS enzyme. Non-limiting examples of mutant E. coli K12 strain BGUS enzymes include those shown in FIG. 1 and in SEQ ID NOs: 2-7. In one embodiment, the mutant BGUS enzyme has an amino acid sequence shown in SEQ ID NO: 2 or SEQ ID NO: 3. In one embodiment, the mutant BGUS enzyme has an amino acid sequence shown in SEQ ID NO: 2 (G559S substitution). In another embodiment, the mutant BGUS enzyme has an amino acid sequence shown in SEQ ID NO: 3 (G559T substitution).

In a preferred embodiment, the preparation containing the BGUS enzyme is substantially free of other non-BGUS proteins. As used herein, “substantially free” refers to less than 5%, preferably less than 3%, even more preferably less than 1% of contamination non-BGUS proteins.

In another preferred embodiment, the preparation containing the BGUS enzyme lacks detectable sulfatase activity. Sulfatase activity can be measured using assays known in the art. For example, sulfatase activity in a formulation can be measured in an enzyme assay using para-nitrocatechol sulfate as the substrate at pH 6.8 at 55 degrees C. for one hour.

### III. Preparation of Formulations

The aqueous and lyophilized formulations of the invention can be prepared using methods well established in the art. Typically, an aqueous formulation is prepared by combining the BGUS enzyme and the excipients at the desired concentrations. A lyophilized formulation can be made by freeze-drying the aqueous formulation using techniques well established in the art. A non-limiting example of suitable conditions for freeze-drying (lyophilizing) of an aqueous solution are set forth in Example 6. All excipients for use in the formulations are commercially available.

Furthermore, it has been discovered that when preparing the aqueous formulation, combining the BGUS enzyme first with the amphoteric compound (e.g., betaine), L-histidine and β-alanine prior to addition of the sugar (e.g., sucrose) results in the most beneficial thermostability and other advantageous properties. Accordingly, in one aspect, the invention provides a method of preparing a β-glucuronidase (BGUS) enzyme formulation comprising:

(a) preparing a solution comprising a BGUS enzyme, an amphoteric compound, L-histidine and β-alanine; and

(b) adding a sugar to the solution prepared in step (a).

In one embodiment, the method further comprises lyophilizing the solution prepared in step (b).

The formulations can be prepared with any of the BGUS enzymes set forth in subsection II above. The formulations can be prepared with any of the excipients and at any of the suitable concentrations set forth above in subsection I. In one embodiment, the formulation (aqueous or lyophilized) is prepared at a higher concentration than the concentration to be used in an enzymatic assay and then the formulation is diluted (e.g., with water) prior to use in the enzymatic assay. For example, a highly concentrated lyophilized formulation can be prepared and packed with instructions for diluting the formulation prior to use in an enzymatic assay. Accordingly, in one embodiment, the invention provides a packaged β-glucuronidase (BGUS) enzyme formulation comprising a container comprising a lyophilized formulation comprising 5 mg/ml BGUS enzyme, 250 mM betaine monohydrate, 50 mM L-histidine, 250 mM β-alanine and 250 mM sucrose; and instructions to dilute the lyophilized formulation to 1 mg/ml BGUS enzyme, 50 mM betaine monohydrate, 10 mM L-histidine, 50 mM β-alanine and 50 mM sucrose before use in an enzymatic assay.

Non-limiting examples of suitable containers for use in a packed formulation include, bottles, tubes, vials, ampules and the like. Preferably, the container is glass or plastic, although other suitable materials are known in the art. Non-limiting examples of suitable instruction media include labels, pamphlets, inserts, and digital media.

### IV. Methods of Using Formulations

The BGUS enzyme formulations of the invention can be used in methods for hydrolysis of glucuronide substrates. These methods can be used, for example, for clinical purposes, for forensic purposes, for industrial manufacturing purposes or for agricultural purposes. These methods are particularly useful for analyzing bodily samples for the presence of drugs through detection of the glucuronide detoxification products of the drugs, e.g., for clinical or forensic purposes. Additionally, beta agonists have been used illegally in meat husbandry, since they can promote muscle growth instead of fat growth in animals (see e.g., J. Animal Sci. (1998) 76:195-207). Thus, the BGUS enzyme formulations also can be used for agricultural purposes in detecting beta agonist residues in meat products.

Thus, in another aspect the invention pertains to a method of hydrolyzing a substrate comprising a glucuronide linkage, the method comprising contacting the substrate with a BGUS enzyme formulation of the invention under conditions such that hydrolysis of the glucuronide linkage occurs.

In one embodiment, the substrate is an opiate glucuronide. Non-limiting examples of suitable opiate glucuronide substrates include morphine-3β-D-glucuronide, morphine-6β-D-glucuronide, codeine-6β-D-glucuronide, hydromorphone-3β-D-glucuronide, oxymorphone-3β-D-glucuronide, and combinations thereof. In another embodiment, the substrate is a benzodiazepine glucuronide. Non-limiting examples of suitable benzodiazepine glucuronide substrates include the glucuronides of oxazepam, lorazepam, temazepam, and alpha-hydroxy-alprazolam. Other suitable substrates include the glucuronides of buprenorphine, norbuprenorphine, 11-nor-Δ9-tetrahydrocannabinol-9-carboxylic acid, testosterone, androsterone, tapentadol, cyclobenzaprine, amitripyline and combinations thereof. In one embodiment, the substrate is a beta agonist (e.g., for meat product analysis). Non-limiting examples of suitable beta agonist glucuronide substrates include clenbuterol, ractopamine and salbutamol.

The methods of the invention can be used on a variety of different bodily samples. Non-limiting examples of suitable bodily samples include blood, urine, tissue or meconium obtained from a subject. For meat product analysis, the bodily sample can be a meat sample. Bodily samples can be obtained, stored and prepared for analysis using standard methods well established in the art.

Following hydrolysis by the enzyme, the cleavage products in the sample can be analyzed by standard methodologies, such as high performance liquid chromatography (HPLC), gas chromatography (GC) and/or mass spectrometry (MS). Such approaches for analysis of bodily samples for the presence of drugs are well established in the art. For example, a completely automated workflow for the hydrolysis and analysis of urine samples by LC-MS/MS, which can be applied using the mutant enzymes of the invention for hydrolysis, is described in Cabrices, O.G. et al., GERSTEL AppNote AN/2014/4-7. Additional liquid chromatography and tandem mass spectrometry (LC-MS/MS) methodologies suitable for use with the invention are described in Sitasuwan, P. et al. (2016) J. Analytic. Toxicol. 40:601-607. Methods for detecting beta agonist residues in meat products using UPLC-MS/MS have also been described (www.waters.com/webassets/cms/library/docs/720004388en.pdf).

The present invention is further illustrated by the following examples, which should not be construed as further limiting. The contents of Sequence Listing, figures and all references, patents and published patent applications cited throughout this application are expressly incorporated herein by reference.

### EXAMPLES

### Example 1: Analysis of Amino Acids as Thermostabilizers

In this example, a panel of amino acids was examined for their effect on the melting temperature of the IMCSzyme® genetically modified (mutant) recombinant E. coli β-glucuronidase enzyme. For all experiments described in the Examples, the IMCSzyme® BGUS enzyme (commercially available from Integrated Micro-Chromatography Systems, LLC) was used in the formulations.

The melting temperature of the enzyme in formulations containing different amino acids was examined using a protein thermal shift assay. For this assay, enzyme protein was purified using standard chromatography techniques to achieve at least >99% purity based on SDS PAGE. Purified recombinant enzyme was initially dialyzed against pure water (18.2 Mohm) with a protein concentration level of at least 2 mg/mL. This stock solution of enzyme in water was mixed 1:1 with various buffer solutions to achieve the final concentration listed in Table 1 below. All samples were then mixed with the dye provided by Protein Thermal Shift™ reagent as specified by the vendor (Life Technologies, CA). This fluorescent dye is activated upon unfolding of the protein. The samples were then placed in a real time PCR machine (StepOnePlus™) using vendor specified ramp rate and temperatures. Protein Thermal Shift Software was used to analyze the melt curves.

The results for the panel of amino acids tested are shown below in Table 1:

TABLE 1

| Amino Acids as stabilizers | | Time in weeks of storage | | |
|---|---|---|---|---|
| | Formulations | 0 | 3 | 7.6 |
| 1 | 50 mM L-Arginine | −4.4 | −30.3 | −29.3 |
| 2 | 50 mM L-Arginine 50 mM | 2.8 | 2.3 | 2.6 |
| | L-Glutamic acid | | | |
| 3 | 100 mM Glycine | −1.3 | −1.3 | −0.6 |
| 4 | 100 mM L-Proline | −0.1 | −0.1 | 1.1 |
| 5 | 100 mM L-Serine | −0.4 | −0.8 | −0.2 |
| 6 | 100 mM L-Argininamide | −22.8 | −25.0 | |
| | dihydrochloride | | | |
| 7 | 100 mM Taurine | −4.1 | −2.6 | −1.6 |
| 8 | 100 mM Diglycine | −4.4 | −3.4 | −1.9 |
| 9 | 40 mM Triglycine | −4.9 | −3.8 | −2.2 |
| 10 | 1% w/v Tryptone | 3.4 | 1.9 | 0.3 |
| 11 | 24 mM L-Histidine | 2.3 | 1.4 | |
| 12 | 100 mM Hydroxyectoine | 0.3 | 0.9 | 0.6 |
| 13 | 100 mM β-Alanine | 3.1 | 2.0 | |

The numerical values shown in Table 1 refer to a melt temperature difference between the enzyme in water and the enzyme in the specified formulation. For example, enzyme with 50 mM L-arginine (formulation #1 in Table 1) showed an immediate decrease in stability as indicated with a decrease of 4.4 deg C. in Tm. After three weeks of storage, the Tm further decreased by 30 deg C. This decrease in Tm value compared to water alone suggested a destabilizing effect of L-arginine. In comparison, the combination of arginine and glutamic acid (formulation #2) increased Tm value by 2.8 deg C. when compared to enzyme in water. This suggested an increase in stability. Thus, arginine alone destabilized the enzyme but in combination with glutamic acid, the two amino acids together showed an increase in melt temperature. However, the combination of arginine and glutamic acid had significantly higher level of aggregation compared to the other excipient formulations (see Example 7).

The formulation containing tryptone (formulation #10) showed an increase in melt temperature. However, this formulation also exhibited a high level of aggregation.

Of all the amino acids tested, only the formulations containing either L-histidine (formulation #11) or β-alanine (formulation #13) showed an increase in the melting temperature of the enzyme while not significantly inducing aggregation compared to the other excipient formulations.

### Example 2: Analysis of Polyols as Thermostabilizers

In this example, a panel of polyol sugars was examined for their effect on the melting temperature of the IMCSzyme® genetically modified β-glucuronidase enzyme, using the protein thermal shift assay described in Example 1.

The results for the panel of polyols tested are shown below in Table 2:

TABLE 2

| | | Storage time (weeks) | | |
|---|---|---|---|---|
| Polyols as stabilizers | | 0 | 3 | 7.6 |
| 14 | 150 mM D-(+)-Trehalose dihydrate | 0.2 | 1.0 | −0.3 |
| 15 | 400 mM Xylitol | 1.8 | 1.2 | 1.8 |
| 16 | 200 mM D-Sorbitol | 2.1 | 1.6 | −0.8 |
| 17 | 400 mM Sucrose | 1.4 | 0.9 | 2.6 |
| 18 | 400 mM Methyl α-D-glucopyranoside | 1.9 | −3.9 | 1.6 |
| 19 | 8% v/v Glycerol 40 mM Lithium chloride | 1.9 | 1.0 | 0.7 |
| 20 | 10% v/v Glycerol | 2.0 | 1.6 | 1.7 |
| 21 | 2% v/v Ethylene glycol | 0.1 | 0.5 | 0.6 |
| 22 | 2% v/v Polyethylene glycol 200 | −3.8 | 0.5 | 0.2 |
| 23 | 1% v/v Polyethylene glycol | −2.9 | −3.8 | |
| | monomethyl ether 550 | | | |
| 24 | 10% v/v Polypropylene glycol P 400 | −9.9 | −7.4 | −4.5 |
| 25 | 5% v/v Pentaerythritol ethoxylate | −0.2 | 0.0 | 0.6 |
| | (15/4 EO/OH) | | | |
| 26 | 2% w/v 1-2-Propanediol | −0.4 | −0.5 | 0.1 |
| 27 | 2 mM (2-Hydroxypropyl)-β-cyclodextrin | 1.3 | 1.9 | 1.5 |
| 28 | 16 mM α-Cyclodextrin | 1.3 | 0.9 | 1.2 |
| 29 | 2 mM β-Cyclodextrin | −10.2 | −2.2 | −2.0 |

While most of the sugars tested as excipients had initially showed an increase in Tm values when compared to water, only two compounds, xylitol (formulation #15) and sucrose (formulation #17) showed consistent increases to Tm. Glycerol (formulation #19 and #20) also improved stability. Complex sugars like (2-Hydroxypropyl)-β-cyclodextrin (formulation #27) and 16 mM α-Cyclodextrin (formulation #28) also had consistent improvements in stabilizing the enzyme by increasing Tm by 1.3 deg C.

### Example 3: Analysis of Salts as Thermostabilizers

In this example, a panel of salts was examined for their effect on the melting temperature of the IMCSzyme® genetically modified β-glucuronidase enzyme, using the protein thermal shift assay described in Example 1.

The results for an initial panel of salts tested are shown below in Table 3:

TABLE 3

| | | Time in weeks of storage | | |
|---|---|---|---|---|
| Salts | | 0 | 3 | 7.6 |
| 30 | 100 mM Sodium malonate pH 7.0 | −2.4 | −1.5 | −0.2 |
| 31 | 100 mM Succinic acid pH 7.0 | −1.0 | −0.9 | 0.2 |
| 32 | 1% v/v Tacsimate pH 7.0 | 1.1 | 1.3 | 1.9 |
| 33 | 15% w/v Trichloroacetic acid | −32.5 | −8.3 | −56.6 |
| 34 | 10 mM EDTA disodium salt dihydrate | −5.4 | −4.5 | −9.2 |
| 35 | 100 mM Guanidine hydrochloride | −4.0 | −4.0 | −7.3 |
| 36 | 100 mM Urea | 1.7 | 0.7 | −0.5 |
| 37 | 100 mM N-Methylurea | 1.9 | −1.0 | −0.4 |
| 38 | 40 mM N-Ethylurea | −65.8 | −4.3 | −1.0 |
| 39 | 10% v/v Formamide | −10.4 | −6.9 | −12.4 |
| 40 | 1% w/v Benzamidine hydrochloride | −3.7 | −4.9 | −14.3 |
| 41 | 100 mM Acetamide | 1.3 | −5.5 | −7.4 |
| 42 | 20 mM MgCl hexahydrate 10 mM | −1.6 | −4.5 | −7.1 |
| | CaCl2 dihydrate | | | |
| 45 | 20 mM CaCl2 hydrate 10 mM | −24.9 | −25.7 | −33.5 |
| | Cobalt(II) chloride | | | |
| 44 | 160 mM Non Detergent | −19.9 | −2.4 | −9.6 |
| | Sulfobetaine 256 (NDSB-256) | | | |
| 45 | 5% w/v 1-Ethyl-3-methylimidazolium | −2.1 | −1.7 | −0.3 |
| | acetate | | | |
| 46 | 5% w/v 1-Butyl-3-methylimidazolium | −23.3 | −26.9 | −2.9 |
| | chloride | | | |
| 47 | 5% w/v Ethylammonium nitrate | −24.4 | −25.4 | −11.3 |
| 48 | 100 mM Ammonium sulfate | −6.7 | −19.0 | −9.8 |
| 49 | 100 mM Ammonium chloride | −8.2 | −20.3 | −9.2 |
| 50 | 100 m M Magnesium sulfate hydrate | −2.2 | −2.9 | −0.6 |
| 51 | 100 mM Potassium thiocyanate | −0.7 | −1.0 | −1.2 |
| 52 | 50 mM Cesium chloride | −10.4 | −7.1 | −8.6 |
| 53 | 100 mM Lithium nitrate | −0.3 | −0.7 | −0.2 |
| 54 | 100 mM Lithium citrate | −2.7 | −3.6 | −3.2 |
| | tribasic tetrahydrate | | | |
| 55 | 50 mM Ammonium acetate | 1.8 | 0.7 | 1.1 |
| 56 | 50 mM Sodium benzenesulfonate | 1.5 | 0.2 | 0.4 |
| 57 | 50 mM Sodium p-toluenesulfonate | 0.4 | 1.3 | 1.4 |
| 58 | 200 mM Sodium chloride | −0.2 | −1.5 | −2.0 |
| 59 | 280 mM Potassium chloride | −0.3 | −1.4 | −2.2 |
| 60 | 200 mM Sodium sulfate decahydrate | 0.4 | −1.7 | −1.4 |
| 61 | 280 mM Lithium chloride | −0.7 | −4.1 | −2.2 |
| 62 | 400 mM Sodium bromide | −0.7 | −1.7 | −1.6 |
| 63 | 20 mM potassium phosphate pH 7.5 | 0.9 | 1.1 | 0.8 |
| 64 | 140 mM Na phos monobasic | −0.7 | −0.6 | 1.1 |
| | 130 mM K phos dibasic | | | |
| 65 | 200 mM Potassium phosphate pH 7.5 | −0.3 | −0.6 | −0.2 |

The majority of the salts that were screened in Table 3 as excipients did not improve the stability of the enzyme except for tacsimate, which is a mixture of titrated organic acid salts.

An additional panel of salts were tested, the results of which are shown below in Table 4:

TABLE 4

| | | Time in weeks of storage | | |
|---|---|---|---|---|
| Salts | | 0 | 3 | 7.6 |
| 66 | 20 mM Kphos pH7.5 10% methanol | −0.1 | −0.2 | 0.0 |
| 67 | 20 mM Kphos pH7.5 5% methanol | 0.1 | 0.2 | 0.4 |
| 68 | 100 mM sucrose 5% methanol | 1.5 | 0.1 | 0.6 |
| 69 | 100 mM sucrose 10% methanol | 0.5 | −0.1 | 2.1 |
| 70 | 100 mM Ethylenediamine dihydrochloride | −22.9 | −33.2 | −25.1 |
| 71 | 100 mM Spermine tetrahydrochloride | −29.2 | | |
| 72 | 100 mM Spermidine | −22.7 | | |
| 73 | 500 mM Trimethylamine N-oxide dihydrate | 3.4 | 2.6 | 2.7 |
| 74 | 5% w/v Tetraethylammonium bromide | −0.7 | −26.8 | −22.6 |
| 75 | 5% w/v Cholin acetate | 0.0 | −0.1 | 1.2 |
| 76 | 500 mM Betaine monohydrate | 2.5 | 1.9 | 0.8 |
| 77 | 100 mM 6-Aminohexanoic acid | 3.3 | 2.3 | 3.4 |
| 78 | 100 mM 5-Aminovaleric acid | 0.4 | 0.8 | 0.2 |
| 79 | 50 mM 4-Aminobutyric acid (GABA) | 1.3 | 1.4 | 0.8 |

Among these salts tested, amphoteric compounds such as betaine monohydrate (formulation #76), 6-aminohexanoic acid (formulation #77), 5-aminovaleric acid (formulation #78) and 4-aminobutyric acid (formulation #79) increased Tm, whereas linear chain amines like spermine (formulation #71) and spermidine (formulation #72) destabilized the enzyme. The majority of ammonium based salts tended to destabilize the enzyme except for trimethylamine N-oxide (formulation #73).

Accordingly, based on the panel of salts tested, the amphoteric compounds were identified as being of greatest interest. While amine oxides such as trimethylamine N-oxide also are suitable for use in the enzyme formulations, the higher cost of such compounds as compared to the amphoteric compounds made them of less interest.

### Example 4: Analysis of Combination Formulations

In this example, based on the initial screens described in Examples 1-3, additional formulations were designed with various combinations of amphoteric compounds, amino acids and sugars to achieve higher Tm values.

Differential scanning fluorescence (DSF), measured using various mixtures of choline, betaine, L-histidine, β-alanine, sugars (sucrose, mannitol, sorbitol and xylitol), was conducted to expand upon the protein thermal shift assays. DSF was carried out using standard methodologies known in the art (see e.g., Ristic, M. et al. (2015) Acta Crytallog. F. Struct. Biol. Commun. 71:1359-1364).

The results for the panel of combination formulations tested are shown below in Table 5:

TABLE 5

| | | | | | Aggre- |
|---|---|---|---|---|---|
| | | Melting | | | gation |
| | Buffer | Onset | Tm 1 | Tm 2 | Onset |
| 80 | 10 mM Choline | 62.5° C. | 71.1° C. | | |
| | hydroxide-Alanine | | | | |
| 81 | 50 mM Cholinehydroxide- | 65.1° C. | 73.5° C. | | 70.6° C. |
| | Alanine | | | | |
| 82 | 100 mM Choline | 66.1° C. | 74.5° C. | | 70.7° C. |
| | hydroxide-Alanine | | | | |
| 83 | 10 mM Choline | 61.5° C. | 70.6° C. | | |
| | chloride-Alanine + | | | | |
| | 10 mM His | | | | |
| 84 | 50 mM Choline | 64.0° C. | 72.5° C. | | 68.5° C. |
| | chloride-Alanine + | | | | |
| | 10 mM His | | | | |
| 85 | 100 mM Choline | 65.1° C. | 73.5° C. | | 67.9° C. |
| | chloride-Alanine + | | | | |
| | 10 mM His | | | | |
| 86 | 10 mM Choline | 62.4° C. | 71.0° C. | | |
| | hydroxide-Alanine + | | | | |
| | 10 mM His | | | | |
| 87 | 50 mM Choline | 65.1° C. | 73.4° C. | | 70.1° C. |
| | hydroxide-Alanine + | | | | |
| | 10 mM His | | | | |
| 88 | 50 mM Choline | 65.6° C. | 73.4° C. | | 70.9° C. |
| | hydroxide-Alanine + | | | | |
| | 10 mM His | | | | |
| | in 20 mM sucrose | | | | |
| 89 | 50 mM Choline | 65.8° C. | 73.6° C. | | 71.1° C. |
| | hydroxide-Alanine + | | | | |
| | 10 mM His | | | | |
| | in 20 mM xylitol | | | | |
| 90 | 100 mM Choline | 66.2° C. | 74.5° C. | | 70.3° C. |
| | hydroxide-Alanine + | | | | |
| | 10 mM His | | | | |
| 91 | 10 mM Choline | 61.0° C. | 69.7° C. | | 67.7° C. |
| | hydroxide-Alanine + | | | | |
| | 10 mM Ala + | | | | |
| | 5 mM His | | | | |
| 92 | 50 mM Choline | 64.0° C. | 72.0° C. | | 67.5° C. |
| | hydroxide-Alanine + | | | | |
| | 50 mM Ala + | | | | |
| | 25 mM His | | | | |
| 93 | 100 mM Choline | 65.7° C. | 73.4° C. | | 67.8° C. |
| | hydroxide-Alanine + 100 mM | | | | |
| | Ala + 50 mM His | | | | |
| 94 | 10 mM Choline | 60.9° C. | 69.6° C. | | 67.1° C. |
| | hydroxyde-AcOH + | | | | |
| | 10 mM Ala + | | | | |
| | 10 mM His | | | | |
| 95 | 10 mM Choline | 62.3° C. | 70.8° C. | | 68.6° C. |
| | hydroxyde-AcOH + | | | | |
| | 50 mM Ala + | | | | |
| | 10 mM His | | | | |
| 96 | 50 mM Ala + 10 | 64.2° C. | 72.6° C. | | 70.5° C. |
| | mM His + 1% | | | | |
| | choline acetate (67 | | | | |
| | mM) | | | | |
| 97 | 50 mM Ala + 10 | 64.5° C. | 73.0° C. | | 70.6° C. |
| | mM His + 1% | | | | |
| | choline acetate (67 | | | | |
| | mM) | | | | |
| 98 | 50 mM Ala + 10 mM His | 60.9° C. | 70.0° C. | | |
| 99 | 10 mM His | 59.7° C. | 69.1° C. | | |
| 100 | 20 mM His | 60.4° C. | 69.5° C. | | |
| 101 | 10 mM His in 20 mM sucrose | | 58.2° C. | 69.0° C. | |
| 102 | 50 mM Ala + 10 mM | 60.1° C. | 69.2° C. | | |
| | His in 20 mM sucrose | | | | |
| 103 | 50 mM Ala + 10 mM | 59.5° C. | 69.2° C. | | |
| | His in 20 mM mannitol | | | | |
| 104 | 50 mM Ala + 10 mM | 59.8° C. | 69.2° C. | | |
| | His in 20 mM sorbitol | | | | |
| 105 | in water (from May 23, 2016) | 59.6° C. | 69.4° C. | | |
| 106 | in water (from May 23, 2016) | 43.1° C. | 53.3° C. | 68.9° C. | |
| 107 | 20 mM sucrose | 43.0° C. | 53.5° C. | 68.3° C. | |
| 108 | 100 mM sucrose | 43.4° C. | 53.8° C. | 68.4° C. | |
| 109 | 20 mM mannitol | 43.0° C. | 53.3° C. | 68.3° C. | |
| 110 | 20 mM sorbitol | 57.6° C. | | 68.5° C. | |
| 111 | 50 mM Choline | 65.1° C. | | 73.0° C. | 70.6° C. |
| | chloride-Alanine + | | | | |
| | 10 mM His in | | | | |
| | 20 mM sucrose | | | | |
| 112 | 50 mM Choline | 64.7° C. | | 72.9° C. | 70.7° C. |
| | chloride-Alanine + | | | | |
| | 10 mM His in | | | | |
| | 20 mM mannitol | | | | |
| 113 | 50 mM Choline | 64.8° C. | | 72.9° C. | 70.6° C. |
| | chloride-Alanine + | | | | |
| | 10 mM His in | | | | |
| | 20 mM sorbitol | | | | |
| 114 | 50 mM Choline | 64.8° C. | | 72.8° C. | 70.2° C. |
| | chloride-Alanine + | | | | |
| | 10 mM His in | | | | |
| | 20 mM xylitol | | | | |
| 115 | 10 mM ChCl:sucrose | 62.0° C. | | 70.7° C. | |
| | (4:1molar ratio, DES) + 50 | | | | |
| | mM Ala + 10 mM His | | | | |
| 116 | 50 mM ChCl:sucrose | 61.0° C. | | 70.7° C. | 69.7° C. |
| | (4:1molar ratio, DES) + 50 | | | | |
| | mM Ala + 10 mM His | | | | |
| 117 | 100 mM ChCl:sucrose | 61.8° C. | | 70.7° C. | 68.1° C. |
| | (4:1molar ratio, DES) + 50 | | | | |
| | mM Ala + 10 mM His | | | | |
| 118 | 20 mM ChCl:sucrose | 62.2° C. | | 70.7° C. | 73.4° C. |
| | (1:1 molar ratio DES) | | | | |
| 119 | 20 mM ChCl:sucrose | 62.3° C. | | 70.8° C. | |
| | (1:1 molar ratio DES) + 50 | | | | |
| | mM Ala + 10 mM His | | | | |
| 120 | 50 mM ChCl:Ala:sucrose | 63.8° C. | | 72.0° C. | 70.5° C. |
| | (4:4:1 mol ratio DES) + | | | | |
| | 10 mM His | | | | |
| 121 | 100 mM ChCl:Ala:sucrose | 64.9° C. | | 73.1° C. | 69.5° C. |
| | (4:4:1 mol ratio DES) + | | | | |
| | 10 mM His | | | | |
| 122 | 10 mM ChCl:sorbitol | 61.8° C. | | 70.5° C. | |
| | (5:2molar ratio, DES) + 50 | | | | |
| | mM Ala + 10 mM His | | | | |
| 123 | 50 mM ChCl:sorbitol | 62.4° C. | | 70.9° C. | 69.7° C. |
| | (5:2molar ratio, DES) + 50 | | | | |
| | mM Ala + 10 mM His | | | | |
| 124 | 100 mM ChCl:sorbitol | 62.1° C. | | 70.7° C. | 67.5° C. |
| | (5:2molar ratio, DES) + 50 | | | | |
| | mM Ala + 10 mM His | | | | |
| 125 | 50 mM ChCl:sorbitol | 61.6° C. | | 70.3° C. | 69.1° C. |
| | (5:2molar ratio, DES) + 10 | | | | |
| | mM His | | | | |
| 126 | 50 mM ChCl:sorbitol | 59.2° C. | | 69.6° C. | 60.2° C. |
| | (5:2molar ratio, DES) | | | | |
| 127 | 10 mM ChCl:mannitol | 62.0° C. | | 70.6° C. | |
| | (5:2molar ratio, DES) + 50 | | | | |
| | mM Ala + 10 mM His | | | | |
| 128 | 50 mM ChCl:mannitol | 62.7° C. | | 71.0° C. | 69.5° C. |
| | (5:2molar ratio, DES) + 50 | | | | |
| | mM Ala + 10 mM His | | | | |
| 129 | 50 mM ChCl:mannitol | 59.9° C. | | 70.1° C. | 68.2° C. |
| | (5:2molar ratio, DES) + 10 | | | | |
| | mM His | | | | |
| 130 | 50 mM ChCl: | 60.1° C. | | 69.9° C. | 62.2° C. |
| | mannitol(5:2molar ratio, | | | | |
| | DES) | | | | |
| 131 | 20 mM xylitol | 58.2° C. | | 68.1° C. | |
| 132 | 10 mM His in 20 mM xylitol | | 52.8° C. | 68.9° C. | |
| 133 | 50 mM Ala + 10 mM | | 52.9° C. | 69.2° C. | |
| | His in 20 mM xylitol | | | | |
| 134 | 50 mM betaine-Ala + 10 mM | | 53.4° C. | 70.7° C. | |
| | His in 20 mM xylitol | | | | |
| 135 | 50 mM betaine-Ala + | 44.7° C. | 53.1° C. | 70.6° C. | |
| | 10 mM His | | | | |
| 136 | 10 mM betaine-Ala + | 43.5° C. | 52.7° C. | 69.2° C. | |
| | 10 mM His | | | | |
| 137 | 50 mM betaine:xylitol | 57.7° C. | | 67.8° C. | |
| | (5:2, mol ration DES) | | | | |
| 138 | 50 mM ChCl:xylitol (5:2, | 63.3° C. | | 71.1° C. | |
| | mol ration DES) + 50 | | | | |
| | mM Ala + 10 mM His | | | | |
| 139 | 50 mM ChCl:xylitol | 59.7° C. | | 69.7° C. | 60.1° C. |
| | (5:2 mol ration DES) | | | | |
| 140 | 50 mM betaine:xylitol | 60.2° C. | | 69.3° C. | |
| | (5:2 mol ration DES) + 50 | | | | |
| | mM Ala + 10 mM His | | | | |
| 141 | 5% glycerol | 43.5° C. | 52.4° C. | 71.0° C. | |
| 142 | 0.1M sucrose (CN | 45.1° C. | 53.5° C. | 69.7° C. | |
| | membrane filtered) | | | | |
| 143 | 0.1M sucrose | 44.8° C. | 53.5° C. | 69.8° C. | |
| 144 | 50 mM Choline | 64.0° C. | | 73.4° C. | 68.2° C. |
| | hydroxide-Alanine + | | | | |
| | 10 mM His | | | | |
| 145 | 50 mM Choline chloride- | 63.8° C. | | 73.1° C. | 69.3° C. |
| | Alanine + 10 mM His | | | | |
| 146 | 50 mM betaine-Ala + | 61.5° C. | | 71.0° C. | |
| | 10 mM His | | | | |
| 147 | 0.1M sucrose | 45.5° C. | 53.7° C. | 69.3° C. | |
| | ( from 2% glycerol) | | | | |
| 148 | 0.1M sucrose | 45.3° C. | 53.7° C. | 69.4° C. | |
| | ( from 2% glycerol) | | | | |
| 149 | 0.1M sucrose | 45.0° C. | 53.4° C. | 69.6° C. | |
| | ( from 10% glycerol) | | | | |
| 150 | 0.1M sucrose | 45.4° C. | 53.6° C. | 69.6° C. | |
| | ( from 10% glycerol) | | | | |
| 151 | 10 mM Choline | 41.3° C. | 48.7° C. | 70.7° C. | |
| | hydroxide-Alanine | | | | |
| 152 | 50 mM Cholinehydroxide- | 64.4° C. | | 73.1° C. | |
| | Alanine | | | | |
| 153 | 100 mM Choline | 41.4° C. | 49.4° C. | 73.9° C. | 68.2° C. |
| | hydroxide-Alanine | | | | |
| 154 | 10 mM Choline | 62.5° C. | | 70.6° C. | |
| | chloride-Alanine + | | | | |
| | 10 mM His | | | | |
| 155 | 50 mM Choline | 64.5° C. | | 72.1° C. | 64.8° C. |
| | chloride-Alanine + 10 | | | | |
| | mM His | | | | |
| 156 | 100 mM Choline | 65.6° C. | | 73.3° C. | 67.4° C. |
| | chloride-Alanine + | | | | |
| | 10 mM His | | | | |
| 157 | 10 mM Choline | 42.7° C. | 50.5° C. | 71.1° C. | 55.5° C. |
| | hydroxide-Alanine + | | | | |
| | 10 mM His | | | | |
| 158 | 50 mM Choline | 64.3° C. | | 73.0° C. | 69.7° C. |
| | hydroxide-Alanine + | | | | |
| | 10 mM His | | | | |
| 159 | 50 mM Choline | 65.6° C. | | 73.4° C. | 71.4° C. |
| | hydroxide-Alanine + | | | | |
| | 10 mM His | | | | |
| | in 20 mM sucrose | | | | |
| 160 | 50 mM Choline | 65.6° C. | | 73.5° C. | 70.6° C. |
| | hydroxide-Alanine + | | | | |
| | 10 mM His | | | | |
| | in 20 mM xylitol | | | | |
| 161 | 100 mM Choline | 64.9° C. | | 74.1° C. | 68.9° C. |
| | hydroxide-Alanine + 10 | | | | |
| | mM His | | | | |
| 162 | 10 mM Choline | 62.0° C. | | 70.3° C. | 68.6° C. |
| | hydroxide-Alanine + | | | | |
| | 10 mM Ala + | | | | |
| | 5 mM His | | | | |
| 163 | 50 mM Choline | 64.0° C. | | 71.9° C. | 67.8° C. |
| | hydroxide-Alanine + | | | | |
| | 50 mM Ala + | | | | |
| | 25 mM His | | | | |
| 164 | 100 mM Choline | 65.5° C. | | 73.2° C. | 68.1° C. |
| | hydroxide-Alanine + 100 mM | | | | |
| | Ala + 50 mM His | | | | |
| 165 | 10 mM Choline | 61.8° C. | | 70.4° C. | |
| | hydroxyde-AcOH + | | | | |
| | 10 mM Ala + | | | | |
| | 10 mM His | | | | |
| 166 | 10 mM Choline | 42.3° C. | 51.2° C. | 71.7° C. | |
| | hydroxyde-AcOH + | | | | |
| | 50 mM Ala + | | | | |
| | 10 mM His | | | | |
| 167 | 50 mM Ala + 10 | 41.5° C. | 49.1° C. | 71.9° C. | 66.7° C. |
| | mM His + 1% | | | | |
| | choline acetate (67 | | | | |
| | mM) | | | | |
| 168 | 50 mM Ala + 10 | 41.9° C. | 49.2° C. | 72.4° C. | 68.3° C. |
| | mM His + 1% | | | | |
| | choline acetate (67 | | | | |
| | mM) | | | | |
| 169 | 50 mM Ala + 10 mM His | 61.5° C. | | 70.0° C. | |
| 170 | 10 mM His | 42.8° C. | 52.1° C. | 69.0° C. | |
| 171 | 20 mM His | 61.2° C. | | 69.3° C. | |
| 172 | 10 mM His in 20 mM sucrose | 60.2° C. | | 68.9° C. | |
| 173 | 50 mM Ala + 10 mM | 60.4° C. | | 69.1° C. | |
| | His in 20 mM sucrose | | | | |
| 174 | 50 mM Ala + 10 mM | 60.2° C. | | 69.1° C. | |
| | His in 20 mM mannitol | | | | |
| 175 | 50 mM Ala + 10 mM | 60.2° C. | | 69.1° C. | |
| | His in 20 mM sorbitol | | | | |
| 176 | in water (from May 23, 2016) | 43.2° C. | 52.1° C. | 69.8° C. | |
| 177 | in water (from May 23, 2016) | 43.3° C. | 52.5° C. | 69.6° C. | |
| 178 | 20 mM sucrose | 58.0° C. | | 68.4° C. | |
| 179 | 100 mM sucrose | 58.7° C. | | 68.5° C. | |
| 180 | 20 mM mannitol | 43.1° C. | 52.6° C. | 69.0° C. | |
| 181 | 20 mM sorbitol | 57.8° C. | | 68.3° C. | |
| 182 | 50 mM Choline | 65.5° C. | | 73.1° C. | 70.9° C. |
| | chloride-Alanine + | | | | |
| | 10 mM His in | | | | |
| | 20 mM sucrose | | | | |
| 183 | 50 mM Choline | 65.2° C. | | 73.0° C. | 70.5° C. |
| | chloride-Alanine + | | | | |
| | 10 mM His in | | | | |
| | 20 mM mannitol | | | | |
| 184 | 50 mM Choline | 65.4° C. | | 73.1° C. | 70.5° C. |
| | chloride-Alanine + | | | | |
| | 10 mM His in | | | | |
| | 20 mM sorbitol | | | | |
| 185 | 50 mM Choline | 65.2° C. | | 72.9° C. | 70.0° C. |
| | chloride-Alanine + | | | | |
| | 10 mM His in | | | | |
| | 20 mM xylitol | | | | |
| 186 | 10 mM ChCl:sucrose | 62.4° C. | | 70.7° C. | |
| | (4:1molar ratio, DES) + 50 | | | | |
| | mM Ala + 10 mM His | | | | |
| 187 | 50 mM ChCl:sucrose | 62.7° C. | | 70.9° C. | 69.4° C. |
| | (4:1molar ratio, DES) + 50 | | | | |
| | mM Ala + 10 mM His | | | | |
| 188 | 100 mM ChCl:sucrose | 62.3° C. | | 70.8° C. | 68.2° C. |
| | (4:1molar ratio, DES) + 50 | | | | |
| | mM Ala + 10 mM His | | | | |
| 189 | 20 mM ChCl:sucrose | 62.3° C. | | 70.7° C. | |
| | (1:1 molar ratio DES) | | | | |
| 190 | 20 mM ChCl:sucrose | 62.6° C. | | 70.8° C. | |
| | (1:1 molar ratio DES) + 50 | | | | |
| | mM Ala + 10 mM His | | | | |
| 191 | 50 mM ChCl:Ala:sucrose | 64.2° C. | | 72.1° C. | 70.1° C. |
| | (4:4:1 mol ratio DES) + | | | | |
| | 10 mM His | | | | |
| 192 | 100 mM ChCl:Ala:sucrose | 65.7° C. | | 73.0° C. | 69.7° C. |
| | (4:4:1 mol ratio DES) + | | | | |
| | 10 mM His | | | | |
| 193 | 10 mM ChCl:sorbitol | 62.6° C. | | 70.6° C. | |
| | (5:2molar ratio, DES) + 50 | | | | |
| | mM Ala + 10 mM His | | | | |
| 194 | 50 mM ChCl:sorbitol | 63.2° C. | | 70.9° C. | 68.8° C. |
| | (5:2molar ratio, DES) + 50 | | | | |
| | mM Ala + 10 mM His | | | | |
| 195 | 100 mM ChCl:sorbitol | 62.5° C. | | 70.6° C. | 67.7° C. |
| | (5:2molar ratio, DES) + 50 | | | | |
| | mM Ala + 10 mM His | | | | |
| 196 | 50 mM ChCl:sorbitol | 62.5° C. | | 70.3° C. | 68.4° C. |
| | (5:2molar ratio, DES) + 10 | | | | |
| | mM His | | | | |
| 197 | 50 mM ChCl:sorbitol | 59.3° C. | | 69.5° C. | 61.1° C. |
| | (5:2molar ratio, DES) | | | | |
| 198 | 10 mM ChCl:mannitol | 62.4° C. | | 70.5° C. | |
| | (5:2molar ratio, DES) + 50 | | | | |
| | mM Ala + 10 mM His | | | | |
| 199 | 50 mM ChCl:mannitol | 62.7° C. | | 70.8° C. | 68.7° C. |
| | (5:2molar ratio, DES) + 50 | | | | |
| | mM Ala + 10 mM His | | | | |
| 200 | 50 mM ChCl:mannitol | 61.5° C. | | 70.1° C. | 68.2° C. |
| | (5:2molar ratio, DES) + 10 | | | | |
| | mM His | | | | |
| 201 | 50 mM ChCl:mannitol | 59.6° C. | | 69.6° C. | 58.9° C. |
| | (5:2molar ratio, DES) | | | | |
| 202 | 20 mM xylitol | 57.7° C. | | 67.7° C. | 42.3° C. |
| 203 | 10 mM His in 20 mM xylitol | | 57.4° C. | 68.6° C. | |
| 204 | 50 mM Ala + 10 mM | 60.4° C. | | 69.0° C. | |
| | His in 20 mM xylitol | | | | |
| 205 | 50 mM betaine-Ala + 10 | 43.6° C. | 53.0° C. | 70.5° C. | |
| | mM His in 20 mM xylitol | | | | |
| 206 | 50 mM betaine-Ala + | 62.0° C. | | 70.4° C. | |
| | 10 mM His | | | | |
| 207 | 10 mM betaine-Ala + | 59.0° C. | | 69.0° C. | |
| | 10 mM His | | | | |
| 208 | 50 mM ChCl:xylitol | 41.8° C. | 52.6° C. | 68.1° C. | |
| | (5:2, mol ration DES) | | | | |
| 209 | 50 mM ChCl:xylitol | | | | |
| | (5:2, mol ration DES) + 50 | | | | |
| | mM Ala + 10 mM His | 61.3° C. | | 70.9° C. | 63.6° C. |
| 210 | 50 mM betaine:xylitol | 37.9° C. | 50.6° C. | 69.7° C. | 46.1° C. |
| | (5:2 mol ration DES) | | | | |
| 211 | 50 mM betaine:xylitol | 58.8° C. | | 68.9° C. | |
| | (5:2 mol ration DES) + 50 | | | | |
| | mM Ala + 10 mM His | | | | |
| 212 | 0.1M sucrose | 43.2° C. | 52.5° C. | 69.7° C. | |
| 213 | 0.1M sucrose | 44.0° C. | 53.0° C. | 69.9° C. | |
| 214 | 5% glycerol | 43.0° C. | 51.4° C. | 68.5° C. | |

The sucrose only formulation (#212 and #213) had an early-onset melting temperature, starting at approximately 43 deg C. The addition of the various salts and the different concentrations increased the melt onset temperature and also increased its overall Tm. Based on the DSF results, sugars alone had early onset of enzyme deformation typically starting around 42 deg C. This early onset of deformation was dramatically shifted with the addition of β-alanine, L-histidine and betaine. Choline salts had varying effects depending on its counter ions and depending on the supply, as well as whether the compound had residual ammonia contaminant that destabilized the enzyme.

Further studies focusing on betaine, L-histidine and β-alanine showed that these three excipients were critical for stabilizing the enzyme against aggregation and increasing melt temperature. Increasing the enzyme concentration from 1-10 mg/mL had no effect in these formulations but higher concentrations of betaine, L-histidine and β-alanine further improved the stability of the enzyme in liquid form.

### Example 5: Analysis of Enzymatic Activity of Aqueous Formulations

In this example, the enzyme activity of various formulations containing excipients identified in Examples 1-4 was examined to determine whether the excipients affected enzyme activity.

To assay enzyme activity, purified enzyme was diluted 200 fold using 20 mM potassium phosphate buffer pH 6.8. The substrate phenolphthalein glucuronide was used at 1 mM concentration. 25 uL of diluted enzyme was mixed with 25 uL of the substrate solution that contained 1 mM phenolphthalein glucuronide. The mixture was mixed on a plate shaker for 30 seconds and incubated at 25 deg C. for 30 minutes. 150 uL of 0.2 M glycine, pH 10.4, was added to the reaction mixture to stop the reaction. The absorbance was measured using a spectrophotometer at fixed wavelength of 540 nm. All samples were measured in triplicates and quantified against a phenolphthalein calibration plot ranging from 1 to 5 micrograms of phenolphthalein.

The results are shown below in Table 6:

TABLE 6

| | | Enzyme |
|---|---|---|
| | | activity |
| Formulations | Tm | (kU/mL) |
| 24 mM L- | 68.1 | 120.7 |
| Histidine | | |
| 100 mM β- | 68.9 | 116.5 |
| Alanine | | |
| 100 mM 6-Aminohexanoic acid | 69.2 | 121.5 |
| 100 mM 5-Aminovaleric acid | 66.0 | 169.0 |
| 50 mM 4-Aminobutyric acid | 66.6 | 200.3 |
| (GABA) | | |
| 400 mM Xylitol | 67.6 | 152.4 |
| 200 mM D- | 67.9 | 154.1 |
| Sorbitol | | |
| 100 mM Sucrose | 67.2 | 162.7 |

The results shown in Table 6 demonstrate that the individual excipients tested (amino acids, amphoteric compounds and sugars) did not significantly affect the activity of the enzyme.

In a second series of experiments, the enzymatic activity of a combination formulation comprising betaine, L-histidine, β-alanine and xylitol in liquid form was tested after prolonged storage (7 days, 26 days or 41 days) at increasing temperatures (4 deg C., 20 deg C. or 37 deg C. ). The results are shown below in Table 7:

TABLE 7

| | Enzyme activity (kU/mL) | | |
|---|---|---|---|
| | After 7 | After 26 | After 41 |
| | days | days | days |
| 4 mg/ml at 4° C. | 338.618 | 337.358 | NA |
| 4 mg/ml at 20° C. | 353.461 | 263.289 | NA |
| 4 mg/ml at 37° C. | 344.54 | 325.479 | 394.059 |
| 1 mg/ml at 4° C. | 93.353 | 76.389 | NA |
| 1 mg/ml at 20° C. | 97.904 | 83.168 | NA |
| 1 mg/ml at 37° C. | 90.285 | 91.481 | 101.56 |

The results in Table 7 demonstrated that the aqueous combination formulation comprising L-histidine, β-alanine, an amphoteric compound (betaine) and a sugar (xylitol) exhibited stable enzyme activity over a prolonged period of time and at increasing temperatures.

### Example 6: Analysis of Enzymatic Activity of Lyophilized Formulations

In this example, combination formulations (comprising L-histidine, β-alanine, an amphoteric compound and a sugar) were prepared and lyophilized, followed by testing of enzymatic activity after different time periods and at different temperatures.

For the lyophilization process, aqueous formulations were re-freezed at −80° C.; Vacuum at 0.2 mBar; Freeze-dried at −50° C. (shelf temperature) −30° C. (sample temperature) for 12-36 hours, then the temperature was raised slowly to −30 to −20° C. (shelf temperature), −20 to −10 to ° C. (sample temperature) and left overnight for 20-48 hours. The next day, the temperature was raised to 4° C. and held for 4 hrs and then raised to 20° C. and held at that temperature for 4-16 hrs. The entire process was 3-6 days total.

The initial formulations tested contained L-histidine, β-alanine and betaine and used xylitol as the sugar excipient for freeze-drying. Enzyme concentration was tested at 1 mg/ml and 4 mg/ml. The enzymatic activity of the lyophilized formulations was compared to the equivalent liquid formulations both immediately after freeze-drying and after 1 week of storage. The results are shown below in Table 8:

TABLE 8

| | | Normalized Enzyme | |
|---|---|---|---|
| | | activity | |
| | | | kU/mL |
| Description | | kU/mL | After 1 week |
| Liquid formula | 4 mg/mL enzyme (betaine, | 97.9 | |
| | histidine, alanine and | | |
| | xylitol) | | |
| Liquid formula | 1 mg/mL enzyme (betaine, | 73.3 | 83.4 |
| | histidine, alanine and | | |
| | xylitol) | | |
| Dried | 1 mg/mL enzyme (betaine, | 63.1 | 80.2 |
| | histidine, alanine and | | |
| | xylitol) | | |
| Dried | 5 mg/mL enzyme (betaine, | 68.5 | 71.1 |
| | histidine, alanine and | | |
| | xylitol) | | |

The results shown in Table 8 demonstrated that the enzyme remained active after freeze-drying, even after 1 week of storage.

The lyophilized formulation was then tested after storage for 24 hours at temperatures between −20 deg C. and 37 deg C. The enzymatic activity for this experiment is shown below in Table 9:

TABLE 9

| | | | |
|---|---|---|---|
| 1 mg/mL enzyme (betaine, | 24 h At −20 C. | 73.8 | 76.0 |
| histidine, alanine and | 24 h At +4 C. | 62.9 | 67.3 |
| xylitol) | 24 h At +20 C. | 0.64 | N/A |
| | 24 h At +37 C. | 0 | N/A |

The results in Table 9 demonstrated that while the formulation remained enzymatically active at lower temperatures, at temperatures at or above 20° C., the enzyme was no longer active. Thus, despite the excipients betaine, L-histidine and β-alanine providing increased enzyme stability in liquid form, the same formulation (containing xylitol as the sugar) did not stabilize the enzyme at high temperatures after the freeze-drying processes.

Further experiments were conducted using different sugar compounds, and different concentrations of sugar, in the formulation to examine whether better protection of the lyophilized formulation against heat inactivation could be achieved.

In a first set of experiments, the betaine, L-histidine, β-alanine and xylitol formulation (referred to herein as Formulation A), was modified by addition of different concentrations of sucrose. Enzyme activity of the liquid versus freeze-dried formulations was tested after 24 hours or 3 days at 37 deg C. The results are shown below in Table 10:

TABLE 10

| | Enzyme activity (kU/mL) | |
|---|---|---|
| | After 24 h at | After 3 |
| | 37 C. | days |
| Formulation A (liquid) | 47.748 | 41.497 |
| Formulation A (dried) | 1.274 | 6.374 |
| Formula A + 10 mM sucrose | 1.511 | 8.415 |
| (dried) | | |
| Formula A + 80 mM suc control | 36.412 | NA |
| (liquid) | | |
| Formula A + 80 mM sucrose | 3.538 | 12.76 |
| (dried) | | |

The results shown in Table 10 demonstrated that the addition of sucrose up to 80 mM ameliorated the freeze dried enzyme activity against higher temperatures but did not confer full protection against heat-inactivation.

Thus, in a second set of experiments, Formulation A was modified by replacing xylitol with various sugars (mannitol, sorbitol, trehalose or sucrose). One formulation, identified here as Formulation B, replaced xylitol with sucrose, while maintaining L-histidine, ⊐-alanine and betaine. Enzymatic activity results for Formulation B with various concentrations of sucrose at high temperatures are shown below in Table 11,

TABLE 11

| | Enzyme activity (kU/mL) | | |
|---|---|---|---|
| | 24 h at | 48 h at | 5 days at |
| | 40° C. | 37° C. | 37 C. |
| Formulation B + 0.1M sucrose (liquid) | 68.0 | NA | NA |
| FB + 0.05M sucrose (dried) | 47.6 | 21.1 | 12.2 |
| FB + 0.1M sucrose (dried) | 58.3 | 23.7 | 19.7 |
| FB + 0.15M sucrose (dried) | 92.2 | 48.5 | 39.4 |
| FB + 0.25M sucrose (dried) | 100.0 | 68.3 | 91.6 |
| FB + 0.5M sucrose (dried) | 89.2 | 70.4 | 104.9 |
| 1% glyc + 0.1M sucrose (liquid) | 83.8 | NA | NA |
| 1% glyc + 0.1M sucrose (dried) | 35.3 | 54.4 | 39.8 |

The results in Table 11 demonstrated that Formulation B containing a minimum concentration of 250 mM sucrose achieved high temperature resistance in both liquid and lyophilized forms. Addition of glycerol and sucrose without the additional Formulation B excipients (L-histidine, β-alanine and betaine) did not stabilize the enzyme at high temperatures. Furthermore, Formulation B also conferred resistance against freeze/thaw, as well as the high temperature resistance, in both liquid and powder form, whereas glycerol or sucrose alone provided no protection against low temperatures in liquid form.

### Example 7: Aggregation Analyses

In this example, various excipients and formulations were tested by differential scanning fluorescence for their effect on aggregation. For the results reported in this example, Tm1 refers to the first peak temperature when the protein unfolds and structural changes are observed based on intrinsic fluorescence shift; Tagg 266 refers to the temperature at which small aggregates are detected; Tagg 473 refers to the temperature at which larger aggregates are detected; Z-ave dia (nm) refers to the diameter of the measured particles in the solution (a larger average diameter refers to more aggregates); and Peak 1 polydispersity % refers to the dispersity index provided by the instrument (UNcle from Unchained Labs) (a lower percentage value indicates a smaller size distribution, which is preferred over a larger percentage value, which indicates a heterogeneous mixture of sizes).

In a first set of experiments, various amino acids (singly and in combination) were examined as compared to Formulation B (containing betaine, β-alanine, L-histidine and sucrose). The results are shown below in Table 12:

TABLE 12

| | | | | | Pk 1 | |
|---|---|---|---|---|---|---|
| | | Tagg | Tagg | Z-Ave. | Poly- | Pk 1 |
| | Tm1 | 266 | 473 | Dia | dispersity | Mass |
| Sample | (° C.) | (° C.) | (° C.) | (nm) | (%) | (%) |
| 1 mg/ml Enzyme in 50 mM Arginine | 36.7 | 44.62 | 36.5 | 78.19 | 24 | 99 |
| 1 mg/ml Enzyme in 50 mM Arginine | 37.7 | 46.04 | 55.6 | 84.84 | 15 | 99 |
| 1 mg/ml Enzyme, 50 mM Arginine, 50 mM | 69.2 | 36.5 | 49.4 | 20.13 | 36 | 100 |
| Glutamic Acid | | | | | | |
| 1 mg/ml Enzyme, 50 mM Arginine, 50 mM | 68.9 | 36.5 | 53.36 | >1000 | 97 | 100 |
| Glutamic Acid | | | | | | |
| 1 mg/ml Enzyme, 50 mM Glutamic Acid | 50.1 | 36.5 | 36.5 | 16.48 | 22 | 100 |
| 1 mg/ml Enzyme, 50 mM Glutamic Acid | 56.6 | 36.5 | 36.5 | 17.09 | 47 | 100 |
| 1 mg/ml Enzyme, 50 mM Glutamic Acid, 24 mM | 39.8 | 36.5 | 36.5 | 16.29 | 50 | 100 |
| Histidine | | | | | | |
| 1 mg/ml Enzyme, 50 mM Glutamic Acid, 24 mM | 37.6 | 36.5 | 36.5 | 16.24 | 40 | 100 |
| Histidine | | | | | | |
| 1 mg/ml Enzyme, 24 mM Histidine | 70.5 | 37.99 | 61.05 | 132.19 | 20 | 100 |
| 1 mg/ml Enzyme, 24 mM Histidine | 70.8 | 38.11 | 63.13 | 111.41 | 16 | 100 |
| 1 mg/ml Enzyme before drying, Formulation B | 71.8 | 66.82 | 72.53 | 9.63 | 26 | 100 |
| 1 mg/ml Enzyme after drying, Formulation B | 72.1 | 68.19 | 71.98 | 10.2 | 33 | 100 |

In the experiment reported in Table 12, the amino acids arginine, glutamic acid and histidine were focused on, since these had showed higher Tm values when tested using the thermal shift assay (see Example 1). However, when tested using differential scanning fluorescence, only L-histidine alone had increased Tm, but L-histidine alone did not prevent aggregation. Only the presence of L-histidine, β-alanine, betaine and sucrose (Formulation B), both before and after drying, showed a high Tm as well as high Tagg 266 and high Tagg 473 values, along with a low polydispersity index, thus indicating that Formulation B prevented aggregation.

In a second set of experiments, various components of Formulation B, alone and in combinations, were tested at various concentrations for their effect on aggregation. The results are shown below in Table 13:

TABLE 13

| | | | | | Pk 1 | |
|---|---|---|---|---|---|---|
| | | Tagg | Tagg | Z-Ave. | Poly- | Pk 1 |
| | Tm1 | 266 | 473 | Dia | dispersity | Mass |
| Sample | (° C.) | (° C.) | (° C.) | (nm) | (%) | (%) |
| Enzyme, Water | 68.7 | 36.5 | 36.62 | 156.06 | 34 | 100 |
| Enzyme, Water | 68.4 | 36.7 | 38.51 | 160.81 | 33 | 100 |
| Enzyme, 100 mM Sucrose | 68.9 | 39.24 | 68.25 | 278.3 | 36 | 100 |
| Enzyme, 100 mM Sucrose | 68.4 | 39.74 | 67.37 | 303.85 | 60 | 100 |
| Enzyme, 50 mM Trimethylamine N-oxide | 68.7 | 39.88 | 77.79 | 240.31 | 32 | 100 |
| Enzyme, 50 mM Trimethylamine N-oxide | 68.6 | 37.61 | 35.65 | 209.6 | 28 | 100 |
| Enzyme, 50 mM Betaine | 68.3 | 41.7 | 67.37 | 227.29 | 41 | 100 |
| Enzyme, 50 mM Betaine | 68.3 | 39.62 | 67.94 | 202.34 | 22 | 100 |
| Enzyme, 25 mM Tris-HCl, 50 mM Betaine, pH 8 | 67.7 | 41.39 | 66.8 | 138.88 | 23 | 99 |
| Enzyme, 25 mM Tris-HCl, 50 mM Betaine, pH 8 | 68.1 | 36.5 | 67.18 | 137.34 | 18 | 99 |
| Enzyme, 50 mM Alanine, 10 mM Histidine | 69.5 | 44.36 | 68.57 | 151.53 | 35 | 100 |
| Enzyme, 50 mM Alanine, 10 mM Histidine | 69.5 | 43.09 | 70.34 | 189.39 | 48 | 100 |
| Enzyme, 50 mM Alanine, 25 mM Histidine | 70 | 39.51 | 70.65 | 153.54 | 19 | 99 |
| Enzyme, 50 mM Alanine, 25 mM Histidine | 70.2 | 52.76 | 70.72 | 152.06 | 44 | 100 |
| Enzyme, 25 mM Alanine, 50 mM Betaine, 10 mM | 69.3 | 43.41 | 68.25 | 188.19 | 48 | 100 |
| Histidine | | | | | | |
| Enzyme, 25 mM Alanine, 50 mM Betaine, 10 mM | 69.2 | 42.08 | 68.44 | 157.33 | 30 | 100 |
| Histidine | | | | | | |
| Enzyme, 50 mM Alanine, 50 mM Betaine, 10 mM | 69.8 | 42.02 | 68.57 | 224.6 | 27 | 100 |
| Histidine | | | | | | |
| Enzyme, 50 mM Alanine, 50 mM Betaine, 10 mM | 69.6 | 40.57 | 68.5 | 202.28 | 33 | 100 |
| Histidine | | | | | | |
| Enzyme, 50 mM Alanine, 50 mM Betaine, 25 mM | 70.3 | 41.58 | 71.03 | 195.81 | 70 | 100 |
| Histidine | | | | | | |
| Enzyme, 50 mM Alanine, 50 mM Betaine, 25 mM | 69.8 | 46.89 | 68.88 | 187.58 | 37 | 100 |
| Histidine | | | | | | |
| Enzyme, 50 mM Alanine, 50 mM Betaine, 10 mM | 69.6 | 66.89 | 71.03 | 95.48 | 23 | 100 |
| Histidine, 50 mM Sucrose | | | | | | |
| Enzyme, 50 mM Alanine, 50 mM Betaine, 10 mM | 69.7 | 66.81 | 70.84 | 82.51 | 21 | 100 |
| Histidine, 50 mM Sucrose | | | | | | |
| Enzyme, 125 mM Alanine, 125 mM Betaine, | 72.8 | 65.41 | 74 | 272.29 | 55 | 100 |
| 25 mM Histidine, 50 mM Sucrose | | | | | | |
| Enzyme, 125 mM Alanine, 125 mM Betaine, | 72.7 | 68.44 | 73.24 | 213.98 | 61 | 100 |
| 25 mM Histidine, 50 mM Sucrose | | | | | | |
| Enzyme, 50 mM Alanine, 250 mM Betaine, 10 mM | 69.6 | 47.83 | 70.91 | 199.3 | 35 | 100 |
| Histidine | | | | | | |
| Enzyme, 50 mM Alanine, 250 mM Betaine, 10 mM | 69.8 | 46.7 | 71.22 | 193.42 | 42 | 100 |
| Histidine | | | | | | |
| Enzyme, 150 mM Alanine, 250 mM Betaine, | 71.7 | 38.65 | 73.43 | 251.2 | 31 | 100 |
| 25 mM Histidine | | | | | | |
| Enzyme, 150 mM Alanine, 250 mM Betaine, | 71.9 | 37.87 | 72.8 | 249.17 | 36 | 100 |
| 25 mM Histidine | | | | | | |
| Enzyme, 250 mM Alanine, 250 mM Betaine, | 73.1 | 36.77 | 74.13 | 261.25 | 46 | 100 |
| 25 mM Histidine | | | | | | |
| Enzyme, 250 mM Alanine, 250 mM Betaine, | 73.4 | 38.8 | 74.89 | 238.89 | 31 | 99 |
| 25 mM Histidine | | | | | | |

The results in Table 13 demonstrated that inclusion of all four components of Formulation B improved the Tagg 266 value as compared to use of less than all four components. Furthermore, increasing concentrations of 3-alanine, betaine and L-histidine improved Tm and Tagg 266 (small aggregates). Sucrose or betaine or histidine/β-alanine improved Tagg 473 (large aggregates) in comparison to water. Overall, incremental benefits to increases in Tagg 266 are observed as betaine, 3-alanine, L-histidine and sucrose are added to the enzyme solution.

In a third set of experiments, the effect on aggregation of adding bovine serum albumin (BSA) or 1,4-dithiothreitol (DTT) to enzyme in Formulation B or in water was examined. For this experiment, Formulation B (FB) contained 50 mM betaine, 50 mM β-alanine, 10 mM L-histidine and 50 mM sucrose. The results are shown below in Table 14:

TABLE 14

| | | | | | Pk 1 | |
|---|---|---|---|---|---|---|
| | | Tagg | Tagg | Z-Ave. | Poly- | Pk 1 |
| | Tm1 | 266 | 473 | Dia | dispersity | Mass |
| Sample | (° C.) | (° C.) | (° C.) | (nm) | (%) | (%) |
| Enzyme in 100 mM sucrose | 67.89 | 71.77 | 74.53 | 127.5 | 29 | 99 |
| Enzyme in water | 68.1 | 72.84 | 73.95 | 95.27 | 31 | 99 |
| Enzyme in water | 68.2 | 72.73 | 73.65 | 135.91 | 33 | 100 |
| Enzyme in Formulation B (FB) | 71.7 | 70.81 | 71.86 | 238.86 | 33 | 100 |
| Enzyme with 0.25 mg/ml BSA in water | 67 | 63.6 | 69.79 | 141.71 | 20 | 99 |
| Enzyme with 0.25 mg/ml BSA in water | 67.5 | 63.34 | 70.82 | 159.79 | 27 | 100 |
| Enzyme with 0.25 mg/ml BSA in FB | 71.9 | 72.54 | 73.47 | 219.8 | 47 | 100 |
| Enzyme with 0.25 mg/ml BSA in FB | 71.8 | 72.55 | 74.86 | 227.13 | 35 | 100 |
| Enzyme with 1 mM DTT in water | 67.5 | 61.67 | 74.49 | 215.72 | 35 | 100 |
| Enzyme with 1 mM DTT in water | 67.6 | 60.38 | 75.17 | 113.48 | 43 | 100 |
| Enzyme with 1 mM DTT in FB | 70.9 | 63.24 | 49.21 | 243.46 | 26 | 100 |
| Enzyme with 1 mM DTT in FB | 70.9 | 68.62 | 74 | 256.1 | 40 | 100 |

The results in Table 14 demonstrated that adding BSA or DTT does not improve the stability of the enzyme. Rather, it is the presence of the excipients in Formulation B that increases Tm and results in higher Tagg values. Adding reducing agents like DTT reduces Tm values and shows aggregation at lower temperatures, as indicated by Tagg 266. BSA also destabilizes the enzyme.

Thus, overall, these experiments demonstrate the beneficial effects of the components of Formulation B (L-histidine, β-alanine, betaine and sucrose) on stabilizing the enzyme preparation and decreasing aggregation of the preparation.

### Example 8: Comparison of Aqueous and Lyophilized Enzyme Formulations

In this example, the ability of aqueous and lyophilized enzyme preparations in Formulation B (50 mM betaine, 50 mM β-alanine, 10 mM L-histidine and 50 mM sucrose) to hydrolyze various glucuronide linkages was compared. Enzyme reactions were carried out under standard conditions. Reaction products can be analyzed using methods well established in the art, such as by liquid chromatography and tandem mass spectrometry (LC-MS/MS) (e.g., as described in Sitasuwan, P. et al. (2016) J. Analytic. Toxicol. 40:601-607). The results for the aqueous and lyophilized enzyme formulations are shown below in Tables 15 and 16, respectively.

TABLE 15

| | Aqueous Enzyme | | |
|---|---|---|---|
| | Theoretical | Calculated | % |
| Compound | Amt | Amt | Difference |
| buprenorphine | 152.539 | 152.476 | −0.04 |
| codeine | 132.206 | 128.56 | −2.76 |
| hydromorphone | 129.841 | 127.917 | −1.48 |
| lorazepam | 135.477 | 154.453 | 14.01 |
| morphine | 129.841 | 123.216 | −5.10 |
| norbuprenorphine | 147.269 | 139.922 | −4.99 |
| oxazepam | 130 | 133.041 | 2.34 |
| oxymorphone | 132.528 | 129.83 | −2.04 |
| tapentadol | 116.934 | 114.907 | −1.73 |
| temazepam | 132.355 | 124.95 | −5.59 |

TABLE 16

| | Lyophilized Enzyme | | |
|---|---|---|---|
| | Theoretical | Calculated | % |
| Compound | Amt | Amt | Difference |
| buprenorphine | 152.539 | 155.505 | 1.94 |
| codeine | 132.206 | 127.479 | −3.58 |
| hydromorphone | 129.841 | 128.937 | −0.7 |
| lorazepam | 135.477 | 155.894 | 15.07 |
| morphine | 129.841 | 122.149 | −5.92 |
| norbuprenorphine | 147.269 | 141.25 | −4.09 |
| oxazepam | 130 | 131.692 | 1.3 |
| oxymorphone | 132.528 | 127.013 | −4.16 |
| tapentadol | 116.934 | 114.554 | −2.04 |
| temazepam | 132.355 | 126.195 | −4.65 |

The results in Tables 15 and 16 demonstrate that the aqueous and lyophilized enzyme preparations in Formulation B exhibit no measurable difference in performance for detecting various glucuronidated drugs. These results were confirmed with urine samples from over 40 different patients, tested for a wide variety of glucuronidated drugs, including opiates and benzodiazepines, which also showed no measureable difference in the performance of the aqueous versus the lyophilized enzyme preparation.

### Equivalents

Those skilled in the art will recognize, or be able to ascertain using no more than routine experimentation, many equivalents of the specific embodiments of the invention described herein. Such equivalents are intended to be encompassed by the following claims.

### Summary of Sequence Listing

| SEQ ID | |
|---|---|
| NO: | DESCRIPTION |
| 1 | Wild type E. coli K12 BGUS full length |
| | amino acid sequence (FIG. 1) |
| 2 | K1S mutant full length amino acid sequence (FIG. 1) |
| 3 | K1T mutant full length amino acid sequence (FIG. 1) |
| 4 | K3 mutant full length amino acid sequence (FIG. 1) |
| 5 | K3Δ1 mutant full length amino acid sequence (FIG. 1) |
| 6 | K3Δ2 mutant full length amino acid sequence (FIG. 1) |
| 7 | K3Δ2S mutant full length amino acid sequence (FIG. 1) |

1. A formulation comprising a β-glucuronidase (BGUS) enzyme, an amphoteric compound, L-histidine, β-alanine and a sugar.

2. The formulation of claim 1, wherein the amphoteric compound is selected from the group consisting of betaine monohydrate, choline salts, betaine salts, 6-aminohexanoic acid, 5-aminovaleric acid and 4-aminobutyric acid (GABA).

3. The formulation of claim 1, wherein the amphoteric compound is betaine monohydrate.

4. The formulation of claim 3, wherein betaine monohydrate is present at a concentration of:
(a) at least 50 mM;
(b) 50 mM-250 mM; or
(c) 250 mM.

5-6. (canceled)

7. The formulation of claim 1, wherein the sugar is selected from the group consisting of sucrose, sorbitol, xylitol, glycerol, 2-hydroxypropyl-β-cyclodextrin and α-cyclodextrin.

8. (canceled)

9. The formulation of claim 7, wherein the sugar is sucrose and sucrose is present at a concentration of:
(a) at least 50 mM;
(b) 50 mM-500 mM; or
(c) 500 mM.

10-11. (canceled)

12. The formulation of claim 1, wherein β-alanine is present at a concentration of:
(a) at least 50 mM;
(b) 50 mM-250 mM; or
(c) 250 mM.

13-14. (canceled)

15. The formulation of claim 1, wherein L-histidine is present at a concentration of:
(a) at least 10 mM;
(b) 10 mM-50 mM; or
(c) 50 mM.

16-17. (canceled)

18. The formulation of claim 1, wherein the BGUS enzyme is a recombinant or mutant BGUS enzyme.

19. (canceled)

20. The formulation of claim 18, wherein the BGUS enzyme is a mutant BGUS enzyme having the amino acid sequence shown in SEQ ID NO: 2 or SEQ ID NO: 3.

21. The formulation of claim 1, wherein the BGUS enzyme is present at a concentration of:
(a) at least 1 mg/ml;
(b) 1-5 mg/ml;
(c) 5 mg/ml.

22-23. (canceled)

24. The formulation of claim 1, which is free of detergents and/or polymers.

25. (canceled)

26. The formulation of claim 1, which is an aqueous formulation or a lyophilized formulation.

27. (canceled)

28. The formulation of claim 1, which comprises a β-glucuronidase (BGUS) enzyme, 50 mM betaine monohydrate, 10 mM L-histidine, 50 mM β-alanine and 50 mM sucrose.

29. The formulation of claim 28, which comprises a BGUS enzyme having the amino acid sequence shown in SEQ ID NO: 2 or SEQ ID NO: 3 at a concentration of 1 mg/ml.

30. The formulation of claim 29, which is an aqueous formulation.

31. The formulation of claim 1, which comprises a β-glucuronidase (BGUS) enzyme, 250 mM betaine monohydrate, 50 mM L-histidine, 250 mM β-alanine and 250 mM sucrose.

32. The formulation of claim 30, which comprises a BGUS enzyme having the amino acid sequence shown in SEQ ID NO: 2 or SEQ ID NO: 3 at a concentration of 5 mg/ml.

33. The formulation of claim 32, which is a lyophilized formulation.

34. A method of preparing a β-glucuronidase (BGUS) enzyme formulation comprising:
(a) preparing a solution comprising a BGUS enzyme, an amphoteric compound, L-histidine and β-alanine; and
(b) adding a sugar to the solution prepared in step (a).

35. The method of claim 34, which further comprises lyophilizing the solution prepared in step (b).

36. A packaged β-glucuronidase (BGUS) enzyme formulation comprising a container comprising a lyophilized formulation comprising 5 mg/ml BGUS enzyme, 250 mM betaine monohydrate, 50 mM L-histidine, 250 mM β-alanine and 250 mM sucrose; and instructions to dilute the lyophilized formulation to 1 mg/ml BGUS enzyme, 50 mM betaine monohydrate, 10 mM L-histidine, 50 mM β-alanine and 50 mM sucrose before use in an enzymatic assay.

37. A method of hydrolyzing a substrate comprising a glucuronide linkage, the method comprising contacting the substrate with the formulation of claim 1 under conditions such that hydrolysis of the glucuronide linkage occurs.

38. The method of claim 37, wherein the substrate is an opiate glucuronide or benzodiazepine glucuronide.

39. (canceled)

40. The method of claim 37, wherein the substrate is in a sample of blood, urine, tissue or meconium obtained from a subject.
